(19) United States
(12) Patent Application Publication
(10) Pub. No.: US 2026/0331242 A1
(43) Pub. Date: Oct. 22, 2026

(54) MEDICAL VISIT RECORDING SYSTEM AND PACIFIER FEATURES

(71) Applicant: Maurice Marcel Garcia, Los Angeles, CA (US)

(72) Inventor: Maurice Marcel Garcia, Los Angeles, CA (US)

(73) Assignee: Maurice Marcel Garcia, Los Angeles, CA (US)

(21) Appl. No.: 19/112,656

(22) PCT Filed: Sep. 18, 2023
(86) PCT No.: PCT/US2023/074448

§ 371 Date: Mar. 18, 2025

Related U.S. Application Data
(60) Provisional application No. 63/376,106, filed on Sep. 18, 2022.

(51) Int. Cl. G16H 15/00; G16H 10/60; G16H 40/67; G16H 80/00

(52) U.S. Cl. CPC G16H 15/00; G16H 10/60; G16H 40/67; G16H 80/00

(57) ABSTRACT

A system for creating a recording of a consultation between a user and a subject, and for transmitting the recording to the subject or to a secure database. The system may be implemented on an application, such as an application for mobile devices or a web application. In a primary context, the consultation is a medical consultation between a medical practitioner and a patient. In this context, the details of a medical visit can be recorded for the patient to better retain and understand complex medical information, and for the physician to have a record to avoid later disputes. The system may be implemented as part of an electronic medical records database.

### STATEMENT REGARDING CROSS-RELATED APPLICATIONS

This application claims priority to U.S. Provisional Patent Application No. 63/376,106, entitled “Medical Visit Recording System,” filed Sep. 18, 2022, the contents of which are hereby incorporated in their entirety.

### BACKGROUND AND SUMMARY OF THE INVENTION

Medical consultations between health care providers such as doctors and their patients are a critical component of health care. Often, serious medical interventions are discussed at these visits, wherein the doctor or other health care provider must communicate complex medical concepts to their patients to aid the patient in making an informed treatment decision. Patients are often required to choose a course of treatment that has attendant risks and downsides. For example, in the context of choosing among oncology treatments or considering surgical interventions, the patient is advised of the attendant risks such as trauma, side effects, and other morbidities. Likewise, the potential poor outcomes and uncertainties inherent to medicine must be addressed.

Meanwhile, in the United States and many other countries, rigorous electronic medical record keeping systems have been put in place. Despite the sophistication of these systems, the record systems currently in place provide, at most, a summary of the interaction between provider and patient that occurs during an office visit. For example, the provider might be able to write notes about the visit, and as shorthand, medical record codes may be selected to summarize what was discussed, the patient's condition, etc. However, these summaries are far from comprehensive and the rich details of the visit are lost. This loss of information creates potential problems for both the doctor and the patient.

For the patient, there is the risk that the complex information communicated by the provider was not fully absorbed or understood, and therefore the patient will not be empowered to make a fully informed decision regarding his or her medical care. For the health care provider, there is the risk that with the volume of patients that are handled and the passage of time, the health care provider may not recall important details of the visit, which details are not captured by the spare medical record that is made under standard practices. In the case of later disputes and allegations of malpractice, there is the danger that the patient will misremember or misrepresent the conversation that occurred between the doctor and the patient, which the doctor will be unable to refute by a spare medical record comprising a written summary.

Standard medical records of visits fail to capture other important aspects of the visit. For example, various nuances that are themselves a form of communication by the provider to the patient cannot be captured by a standard medical visit record. Examples include the provider's tone, and how the provider chooses to illustrate a particular point. A provider may channel their caring, or urgency, seriousness, and/or self-confidence, by the tone they use when conversing with a patient. The aforementioned constitute “meta-data” that cannot be captured effectively by a transcription of a conversation. Such meta-data can also serve to support a provider's assertion that they attempted to give an unbiased, and/or comprehensive review of a treatment, or the risks/benefits of that treatment. Similarly, such meta-data could support a patient's assertion that their provider was dismissive of their complaints, or “gaslighted” them, for example, as described in “Feeling Dismissed? How to Spot ‘Medical Gaslighting’ and What to Do About It,” by Christina Caron, New York Times, Jul. 29, 2022.

In contrast to a standard written medical record, an audio recording can capture all of the aspects of a medical visit. Accordingly, there is a need in the art for a facile and convenient means of creating a medical record that comprises a recording of the conversations that occurred during an office visit or other consultation. There is a need in the art for a means of sharing this recording with the patient, and storing the recording in a secure storage or as part of the patient's medical record.

Lastly, just as a recorded conversation affords a patient the opportunity to more fully process and benefit from the valuable contents within the recorded conversation, the recording itself is even more useful to patients when it can be paired with auxiliary materials related to the conversation itself: illustrations and/or photographs that explain or illustrate what was discussed, and published texts that expound on points made by the provider.

The foregoing needs in the art are fulfilled by the inventions described herein. The scope of the invention encompasses a system for the creation, storage, and sharing of an audio or video recording of a provider-patient interaction. In a primary embodiment, the system is configured as an application running on a smartphone or other mobile device which enables the provider to record the visit, share the recording with the patient, and store the recording in a secure site for later retrieval if needed.

The various elements of the system in association with various embodiments are described next.

### SUMMARY OF THE INVENTION

In one aspect, provided herein are systems for recording an interaction between two parties, transmitting the recording to one or both parties, and storing the recording of the interaction for later retrieval. In one implementation, the interaction is a medical visit and the parties are a medical provider and patient. In other instances, the interaction may be any sort of consultation between a service provider and a customer.

The operations of the system are coordinated or carried out in part by the operation of a software application, for example, an application residing and operating on the mobile device, comprising a consultation recording and transmission application. In one embodiment, the consultation recording and transmission application resides and operates entirely on the user's device. In one embodiment, the consultation recording and transmission application is a distributed application residing in part on the user's device and in part on a remote system. In one embodiment, the consultation recording and transmission application is a web application residing and operating on a remote system and accessed by a web browser application operating on the user's device.

In one aspect, the consultation recording and transmission application engages recording hardware elements present on the user's device to capture a recording of the consultation. The recording data is stored in a file, which is subsequently transmitted to one or more selected recipients. In one embodiment, the recipient is the user. In one embodiment, the recipient is the subject of the interaction with the user. In one embodiment, the recipient is a secure repository such as a medical records database.

The recording data file is sent in a message. In one aspect, the message is an email message. In one aspect, the system provides tools and features for the user to augment the message with additional information in addition to the recording data file, including additional information about the interaction, supplementary material pertinent to the interaction, follow up actions and calendar items, and other functions.

In one aspect, the system is configured to maintain data security and patient privacy by functions that securely store the recording and, after transmission, remove the recording from the device on which it was recorded.

In one aspect, the scope of the invention encompasses methods of using the system to perform the functions of recording an interaction, transmitting the recording, appending additional materials, and, in some embodiments, maintaining data security by deletion of information from the device once it has been transmitted.

In one aspect, the systems are utilized in combination with one or more analysis applications that analyze the recording file data and provide analysis outputs. In one implementation the analysis application provides analysis of the recording data in real-time, as the recording is being captured. In one implementation, the analysis is applied following completion of the recording. Analysis applications may provide written transcripts of oral interactions, translation of oral interactions to different languages, and other analyses.

In one aspect, the scope of the invention encompasses devices configured to perform primary functions of the method, or to perform the methods in coordination with remote systems, such as medical records systems.

These and other aspects of the invention are next described in detail.

### BRIEF DESCRIPTION OF THE DRAWINGS.

FIG. 1 is a diagram of device elements that may be present in a device configured to perform certain device-residing functions of the inventive methods. The device 100 comprises various hardware elements, including network elements 101, user interface elements 102, recording elements 103, and one or more processors 104. The device further comprises one or more data storage elements 105. Applications stored in the data storage element may include a consultation recording and transmission application 106, a communication application (e.g., email application) 107, a web browser application 108, and one or more analysis applications 109. Data storage may comprise a secure folder 110. Data storage may comprise one or more consultation data files 111, one or more recording files 112, one or more analysis files 114. The data storage element may comprise one or more ancillary files 113.

FIG. 2 is a diagram of a remote system of the invention and various elements of the remote system, which perform various functions in certain methods of the invention. The remote system 200 comprises various hardware elements, including network elements 201, one or processors 202. The remote system will comprise one or more data storage elements 203. Applications stored in the data storage element may include a consultation recording and transmission application 204, a communication application (e.g., email application) 205, a database 206, one or more analysis applications 207. Data storage may comprise one or more consultation data files 208, one or more recording files 209, one or more analysis files 211. The data storage element may comprise one or more ancillary files 210.

FIG. 3 depicts network connections among various elements utilized in the methods of the invention, including a device 100, a remote system 200, a private network 302, a public network 301, a communication server such as an email server 303, a recipient server, such as a secondary email server 304, and one or more remote servers 305.

FIGS. 4A and 4B are flow charts depicting a general process of the invention. In FIG. 4A, the initial steps of the process are depicted: the user initiates a consultation session 401 by entering command on a device. This command directs a consultation recording and transmission application (as a local application, a distributed application comprising a client-side application in network connection with a server-side application) or a web application operating through a web browser application) to create a consultation data file 402. The user enters or selects consultation data, including a recipient address, which data is written to the consultation data file 403. By the device, the user enters a command to initiate a recording 404. In response, the consultation recording and transmission application creates a recording file 405 and the recording elements of the device are engaged and commence capturing recording data, which is written to the recording file. The user, by the device, subsequently enters a command to terminate the recording 407. Continuing in FIG. 4B, the next steps of the process are depicted: the user, by the device, enters a command to initiate a message 408. A communication application is engaged and a message file is created and is populated with data from the consultation data file and recording data from the recording file 409. The user, optionally, by the device, enters or selects ancillary material which is added to the message file 410. The user then, by the device, enters a command to send the message 411. The communication application achieves the sending of the message to selected recipients 412. Optionally, the consultation file data and recording file data are then stored or erased 413.

FIGS. 5A, 5B and 5C are flow charts depicting a process of the invention performed using a local application residing on a user's device. In FIG. 5A, the initial steps of the process are depicted: by the device the user enters a command to open the local consultation recording and transmission application residing on the user's device 501. The user initiates a consultation session 502 by entering command on the device. This command directs the consultation recording and transmission application to create a secure folder in a data storage element of the device 503. Additionally, a consultation data file is created 504 and is stored in the secure folder. The user enters or selects consultation data, including a recipient address, which data is written to the consultation data file 505. By the device, the user enters a command to initiate a recording 506. In response, the consultation recording and transmission application creates a recording file 507 in the secure folder. Continuing the process in FIG. 5B: the recording elements of the device are engaged and commence capturing recording data, which is written to the recording file 508. The user, by the device, subsequently enters a command to terminate the recording 509. The user, by the device, enters a command to initiate a message 510. A communication application, for example, an email application, is engaged and a message file is created and is populated with data from the consultation data file and recording data from the recording file 511. The user, optionally, by the device, enters or selects ancillary material which is added to the message file 512. The user then, by the device, enters a command to send the message 513. The communication application achieves the sending of the message to selected recipients 514. Continuing in FIG. 5C, the next steps of the process are depicted: the consultation recording and transmission application displays a menu for the user wherein they may elect to terminate the session 515. The user may enter a command by the device to terminate the session 516, whereupon the consultation recording and transmission application directs that the secure folder and its contents are erased 517.

FIGS. 6A and 6B are flow charts depicting a process of the invention performed wherein the user engages a device to perform certain steps of the process of the invention by a remote system. In this implementation, a remote system comprising elements of FIG. 2 is engaged by a device, wherein a consultation recording and transmission application operating on the remote system is engaged by (a) a locally-installed consultation recording and transmission application or (b) a web application, operating on the device. In FIG. 6A, the initial steps of the process are depicted: by the device the user enters a command to engage the remote system. A network connection, e.g., a secure network connection is established between the device and the remote system 602. The user initiates a consultation session 603 by entering command on the device. This command directs the consultation recording and transmission application residing on the remote system to create a secure folder in a data storage element of the remote system 604. The user enters or selects consultation data, including a recipient address, which data is written to the consultation data file 605. By the device, the user enters a command to initiate a recording 606. In response, the consultation recording and transmission application creates a recording file 607 in the data storage element of the remote system. Continuing the process in FIG. 6B: the recording elements of the device are engaged and commence capturing recording data, which recording data is transmitted by the device via the network connection and is written to the recording file residing on the remote system 608. The user, by the device, subsequently enters a command to terminate the recording 609. The user, by the device, enters a command to initiate a message 610. A communication application, for example, an email application, residing on the remote system is engaged and a message file is created and is populated with data from the consultation data file and recording data from the recording file 611. The user, optionally, by the device, enters or selects ancillary material which is added to the message file 612. The user then, by the device, enters a command to send the message 613. The communication application achieves the sending of the message to selected recipients 614.

FIGS. 7A, 7B, and 7C depict a general process of the invention with additional features, including the display of guide material, the ability to enter notes during the recording session, and the use of analysis applications. In FIG. 7A, the initial steps of the process are depicted: the user initiates a consultation session 701 by entering command on a device. This command directs a consultation recording and transmission application (as a local application, a distributed application comprising a client-side application in network connection with a server-side application) or a web application operating through a web browser application) to create a consultation data file 702. The user enters or selects consultation data, including a recipient address, which data is written to the consultation data file 703. By the device, the user enters a command to initiate a recording 704. In response, the consultation recording and transmission application creates a recording file 705 and the recording elements of the device are engaged and commence capturing recording data, which is written to the recording file 706. During the recording session, the consultation recording and transmission application displays guide material for the user 707. Continuing the process in FIG. 7B, the user is also presented with fillable boxes or selectable items which enable the user to input additional data about the consultation, which data is written to the consultation data file 708. Optionally, an analysis application, for example, residing on the drive, on a remote system, or accessed from a third party server by network connection, is engaged and performs an analysis of the recording data, the output of which analysis is displayed for the user 709. The user, by the device, subsequently enters a command to terminate the recording 710. Optionally, an analysis application is engaged, for example, residing on the device, on a remote system, or accessed from a third party server by network connection, and performs an analysis of the recording data, which results in the output of one or more analysis files 711. The analysis file may be transmitted to the user's device, or may reside on the remote system. The user, by the device, enters a command to initiate a message 712. A communication application is engaged and a message file is created and is populated with data from the consultation data file, a file comprising the recording data, and the one or more analysis files 713. Continuing in FIG. 7C, the next steps of the process are depicted: the user may, optionally, enter or select ancillary material for inclusion in the message 714. The user subsequently, by the device, enters a command to send the message 715. The communication application achieves the sending of the message to selected recipients 716. Optionally, the consultation file data, the recording data, and the one or more analysis files are then stored or erased 717.

FIGS. 8A, 8B and 8C are flow charts depicting a process of the invention performed using a local application residing on a user's device. In FIG. 8A, the initial steps of the process are depicted: by the device the user enters a command to open the local consultation recording and transmission application residing on the user's device 801. The user initiates a consultation session 802 by entering command on the device. This command directs the consultation recording and transmission application to create a secure folder in a data storage element of the device 803. Additionally, a consultation data file is created 804 and is stored in the secure folder. The user enters or selects consultation data, including a recipient address, which data is written to the consultation data file 805. By the device, the user enters a command to initiate a recording 806. In response, the consultation recording and transmission application creates a recording file 807 in the secure folder. Continuing the process in FIG. 8B: the recording elements of the device are engaged and commence capturing recording data, which is written to the recording file 808. During the recording session, the guide materials are displayed for the user on the display of the device 809. The user is also presented with fillable boxes or selectable items which enable the user to input additional data about the consultation, which data is written to the consultation data file 810. The user, by the device, subsequently enters a command to terminate the recording 811. Following termination of the recording, an analysis application is engaged, for example, an analysis application residing on the device, on a remote system, or accessed from a third party server by network connection, and performs an analysis of the recording data, which results in the output of one or more analysis files 812, which are stored in the secure folder. The analysis application may be a voice-to-text analysis application which analyzes the recording data and generates a transcript and/or translation file of the consultation. The user, by the device, enters a command to initiate a message 813. A communication application, for example, an email application residing on the device, is engaged and a message file is created 814. Continuing the process in FIG. 8A: an message template file is retrieved and the message file is populated with data from the template file, the consultation data file, recording data from the recording file, and the one or more analysis files (e.g., transcripts) 815. The user, optionally, by the device, enters or selects ancillary material which is added to the message file 816. The user then, by the device, enters a command to send the message and the communication application achieves the sending of the message to selected recipients 817. The consultation recording and transmission application displays a menu for the user wherein they may elect to terminate the session 818. The user may enter a command by the device to terminate the session 819, and upon receipt of such command, the consultation recording and transmission application directs that the secure folder and its contents are erased 820.

FIGS. 9A, 9B, 9C, 9D, 9E, 9F, 9G, 9H, 9I, 9J, 9K, and 9L depict screenshots from an exemplary embodiment of an application performing certain inventive methods disclosed herein by a consultation recording and transmission application residing on a device, as described in Example 1.

### DETAILED DESCRIPTION OF THE INVENTION

### Part I. Elements Of The System

### Part I. A-General Elements

The inventions disclosed herein will encompass the various methods disclosed herein. Each such method is performed by a user operating a device, wherein the device is utilized to operate various applications and hardware elements to achieve the functions of the invention. The scope of the invention further encompasses devices configured to carry out the foregoing methods. These methods and devices comprise, or comprise the use of, certain general elements known in the computing arts, as follows.

Local vs. Remote. Reference will be made herein to local and remote elements. Such references are made with respect to the user and the device operated by the user, wherein elements of, and actions performed by, the device are local, and elements and actions performed outside the device, for example in network connection to the device, are referred to as remote. Software applications may be described herein as residing on a device. An application residing on a device means that the application is stored in a data storage element of the device, and the application operates by the operation or one or more processors of the device.

Systems. In one aspect, the inventions described herein encompass systems. Systems comprise a plurality of functional elements that interact to achieve the functions of the invention. In the context of the invention, systems may comprise elements that operate together to achieve the recording of an interaction, the transmission of that recording to selected parties and storage destinations, the optional augmentation of the recording with additional materials, and the maintenance of data security and privacy by erasing recordings following transmission. The systems of the invention may comprises multiple elements which are configured to be operated together to perform the claimed functions set forth herein. System components may include any number of physical components, e.g., electronic devices and computer processors.

Elements of the system will be referred to in the singular, however, unless specified otherwise, it will be understood that the functions of a recited element may be carried out by two or more components working together to achieve a single function, for example, multiple physically separate elements may operate in tandem to perform a recited operation. For example, reference may be made to an operation being carried out by “a processor,” however, it will be understood that multiple processors working together, for example, in parallel, may achieve the recited action or function. Remote and local elements may cooperate to perform a recited function.

Processors. The systems, methods, and devices described herein encompass computer hardware elements and the operation thereof, various computer operations, user-computer interfaces and interactions, and computer-implemented methods. Such operations may be carried out by appropriately instructed and operated general-purpose computer processors and/or special-purpose processor elements. The systems, methods, and devices of the invention are not limited to the use of any one type of processor, software architecture, operating system, connectivity, or network. It is recognized by one of skill in the art that there are multiple programming options for achieving the operations of the systems described herein, including a variety of operating systems, (e.g. iOS, WINDOWS™, LINUX™ and others) and programming languages. The operations of the inventive methods described herein may be carried out by any type or combination of processing circuits, integrated circuits, chips, or systems capable of executing program instructions.

Data Storage. The devices and systems described herein will comprise one or more data storage elements. A data storage element may comprise any computer data storage device capable of receiving and storing data in a format that can be accessed and read out. In some embodiments, the data storage element comprises any non-volatile hard drive, solid-state drive, or hard disk, for example, magnetic or optical data storage devices. Data storage elements may store applications, files, and folders wherein multiple files are stored. Data storage may comprise a database, i.e., data stored in a structured format that may be queried and accessed by suitable protocols. In some embodiments, the data storage comprises elements for storage of data comprising working memory or volatile storage.

Applications. The system of the invention may utilize one or more application elements. Applications are programs that are executable on a computing device. Applications may be configured as software, i.e. machine-readable instructions written on a tangible or non-transitory processor-readable medium storing and comprising processor-readable instructions which, when executed, direct a processor, and/or hardware elements controlled thereby, to perform one or more operations. The applications of the invention may implement a series of steps configured to carry out the various operations of the methods of the invention. In one embodiment, the application is configured as a software application, for example, an “app” which is operated by the user on a device. The software of an application may be stored locally on a device or may be run on one or more servers remote from the device by means of a network connection.

In one embodiment, the invention encompasses the use of a software application configured to operate on a device to perform certain actions of the inventive methods described herein by receiving user inputs and responding thereto by directing the operation of other applications and of hardware elements to achieve the inventive functions. Such application will be referred to herein as a or consultation capture and transmission application and will be described in more detail herein.

Network. The systems of the invention will encompass one or more networked elements, e.g. elements which access a network and communicate with other elements by network connections, as known in the art. In some embodiments, the network is a public networks, for example, the Internet. In some embodiments, the network is a private network. In some embodiments, the network is a virtual private network. The networks may comprise any of wired or wireless connection, e.g. Wi-Fi, Bluetooth, cellular networks, ethernet connections, or wired networks, a local area networks, a wide area network, or other networks as known in the art. Network connections may be facilitated by any suitable hardware elements, such as wireless antennae, fiber optic cables, routers, modems, and other hardware elements and their drivers and associated network and communication applications.

In various embodiments, the methods disclosed herein utilize network connections, for example, to transmit data from a device to a remote system, or to transmit messages to specified recipients. Network connections may comprise secure network connections, for example, such as: access controlled connections, for example, utilizing Identity and Access Management (IAM) or Role-Based Access Control (RBAC); VPN's, such as remote-access VPN's; Zero-trust network access tools; and others known in the art.

Computer Files. The systems of the invention will create, store, and transfer any number of computer files, also referred to as data files or “files.” A computer file, as known in the art comprises data stored on a tangible or non-transitory computer-readable medium. In some implementations, the computer file is encrypted, i.e. securely formatted for selective access by users employing a key, security token, or other encryption tool.

In some embodiments, the computer file is an audio file. An audio file comprises any audio or sound data. The audio file may be of any format, for example, MP3, WAV, AIFF, AU, FLAC, ape, TTA, WMA Lossless and other formats known in the art. In one embodiment the computer file is a video file. Exemplary video formats include MP3 audio files, MP4, .MOV, WMV, AVI and other formats known in the art.

Devices. The systems of the invention may be carried out on a user's device such as a smartphone, tablet, laptop, desktop computer or other such apparatuses comprising multiple integrated devices (hardware elements) and associated software for performing various actions. In a primary embodiment, all or some portion of the processes described herein are carried out on a smartphone. Exemplary smartphones include the Apple IPHONE™ and the Samsung GALAXY™, etc. Devices will comprise additional elements, such as one or more processors configured to operate the device, network elements, displays, user interface elements, data storage elements, and recording elements, as described below.

Network Elements. The user device will comprise network elements, comprising hardware and associated software drivers and controllers thereof, for networked communication with remote servers such as email servers or medical record portals, the internet, etc. Exemplary network hardware elements include, for example, transmitting and receiving antennae for communicating with a cellular network, Bluetooth network, or Wi-Fi network, modems, and other wireless and wired connections.

Recording Elements. The device will comprise one or more hardware elements for capturing, i.e. recording, an audio or video file. In the case of audio files, the hardware element will comprise one or more microphones. In the case of video files, hardware for video acquisition may include, for example, microphones and digital camera modules, for example, embedded in mobile phones, tablets, or laptop computers. The recording elements may also comprise peripheral elements in wired or wireless connection with the device, such as microphones, cameras, and other elements, for example wireless (e.g. Bluetooth connected) elements and/or wired elements such as plug-in webcams, etc.

User Interface Elements. The user device will comprise a user interface, comprising a combination of hardware elements and associated software drivers and controllers thereof, which combination of elements work together to enable the user to operate the device and software programs operated thereby. The user interface elements comprise any combination of elements which enable output to be displayed and for inputs to be received by the user to operate the system.

A key user interface element is a display, e.g. a visual display which can be observed or read by the user. Outputs of the applications described herein may be presented on the display. For example, displayed content may be referred to as a “page” or a “screen,” for example, a “home screen” or “home page” as found on a webpage or app. Displayed screens may show any content required for operation of the system, including for example, graphics, drop-down menus, and displayed buttons that may be engaged to initiate functions, or to link to other pages. The user interface may also comprise one or more speakers for playback of audio data. Tactile outputs, such as braille displays may also be employed.

The user interface will comprise elements that enable the user to make inputs. Exemplary input elements include touchscreens and other touch-sensitive surfaces overlaid on a display for detecting touch-based inputs, for example, with a finger or pen. The input elements may comprise displayed buttons, menus, and other items clickable or selectable by the user's selection, for example by a mouse or touchscreen input. The input elements may comprise text box fields into which a user may enter data by typing. In one embodiment, a fillable text box is presented on a page, and if selected or moused over, a displayed keyboard element is presented to the user enabling them to type inputs to the text box. In some embodiments, the input comprise external peripherals, such as a mouse, keyboard, pen or like element. Exemplary input devices may include, for example, physical buttons, touchscreens, mouse interfaces, pen tool interfaces, keyboard interfaces, and voice activated controllers. In a primary implementation, the user interface comprises a touchscreen.

Reference will be made herein to users “entering or selecting data.” Entering data encompasses the user inputting a data string, for example, by typing the data into a field such as a text box, via displayed or physical keyboard, or by voice-to-text conversion tools. Selection encompasses the user clicking, mousing over, or otherwise indicating the selection of a displayed item, wherein data associated with the chosen item may be retrieved or noted. For example, a user might be presented with a displayed box for entering a name, and the name entered may be stored in a data file, for example, a “name” field in the data file. A user may be presented with a displayed menu of items, wherein checking or choosing the item causes data indicating the selected item to be recorded in a data file.

Web Browser. Certain implementations of the invention encompass the use of a web browser. A web browser is an application which enables a user to access content on a remote server, e.g. a web server. For example, content may be accessed by entering a Uniform Resource Locator (URL) identifying the remote web server to be contacted by the web browser. The web browser opens a network connection with the remote server, typically using HyperText Transfer Protocol (HTTP), whereupon content is retrieved from the web server and rendered (displayed) for the user. In one implementation, the scope of the invention encompasses the use of applications comprising web applications, wherein the web browser, by an accessed web page, acts as an interface to access and operate a remote application. Inputs made in the webpage are transmitted to the remote server by the network, which may perform any number of functions such as receiving data (e.g. uploaded or entered data), processing requests and providing process results, and providing data (e.g. displayed or as a download).

Communication Application. The methods of the invention encompass the use of communication applications for the transmittal of recorded consultations to specified recipients. The scope of the invention encompasses any communication application to transmit a message, wherein the message comprises one or more recipients to which the message is sent, and a body comprising data and linked or embedded files.

In a primary embodiment, the communication application is an electronic mail, a.k.a. email, application which utilizes email protocols known in the art to transmit email messages over a network. The email application may reside on the user's device, or may be a remote application accessed by a network connection. For example, in one implementation, the email application utilizes a sending server, typically an SMTP server to route the message, via a series of routers, to a recipient email server, wherein it can be retrieved by a specified recipient. In some implementations, the sending server is local, for example, residing on a user's device, i.e. the user's device is configured as a client email server. In a more typical implementation, the sending server is remote to the user's device and is accessed by a network connection. Exemplary email applications include Microsoft OUTLOOK™ mail, Apple MAC MAIL™, Beta GMAIL™ and others known in the art.

In a primary implementation, the methods of the invention encompass the transmittal of email messages to one or more recipients. The email message will comprise a header. The header will comprise multiple fields, including recipient email address(es), and a title. The email message will comprise a body, comprising text or other data. The email message may comprise one or more attachments, i.e. files that are included in (embedded) or linked to the email message, for example, wherein the email message comprises a reference to the file and the file is sent together with the email message.

Certain embodiments of the invention encompass the use of email templates. Email templates typically comprise HTML files that define the structure and content of an email message, and may include fillable or merge fields, comprising placeholders, which can be populated with data from a record.

In alternative implementations, the communication application may comprise any other communication tool, for example, short message services (SMS), multimedia message services (MMS), or other communication applications known in the art. In some implementations, a private network connection is utilized to transmit messages. In some embodiments, the communication application comprises a client VPN application residing on a device that enables connection to a remote VPN server. In some embodiments, the communication application comprises a remote VPN server, for example, operating on a remote system or third party server, which facilitates secure communication with client devices.

The transmission may be by any suitable means for the transfer of secure files. In one embodiment, the recording file is sent as an encrypted email attachment to a destination email service where it can be indexed and securely stored. In other embodiments, the app is configured to send the recording file directly to a remote server by transmission protocols such as SSH File Transfer Protocol (SFTP), as known in the art, built on the Secure Shell (SSH) transport systems which utilizes secure channels for file transfer; File Transfer Protocol Secure (FTPS), as known in the art, built on the FTP protocol with Transport Layer Security, which secures the connections and transport channel; and Applicability Statement 2 (AS2) protocol known in the art, which encrypts the files for secure transit using S/MIME cryptographic tools.

Encryption. Certain processes of the invention encompass the creation and transmission of encrypted files or data. Encryption may be by any means known in the art. Common encryption tools include PGP, AES, RSA, Triple DES, and others known in the art. In some embodiments, recording data captured by a device is transmitted to a remote system in an encrypted form. In some embodiments, encrypted email files are sent. Encrypted email transmission may utilize tools known in the art, such as public key encryption schemes, for example, S/MIME and PGP/MIME protocols. HIPPA-compliant email transmission may be utilized in various embodiments of the invention. Exemplary HIPPA-compliant email services include, for example, EGRESS™, LUXSCI™, BARRACUDA™, and others. In the case of email transmissions, the body of the email may be encrypted, and/or attachments, such as files comprising recording data, may be encrypted.

Electronic Medical Record Systems. Certain embodiments of the invention are directed to coordination with remote systems. In some embodiments, the remote system is a electronic health record (EHR) systems, for example, electronic medical record (EMR) systems. EHR and EMR system comprise electronically stored information about a patient, including, for example, medical records, health data, descriptions of services provided and other personal health information for a plurality of subjects. Exemplary EMR systems include the EPIC™, PRAXIS™, and CERNER™ systems.

Electronically stored health data may be accessed and retrieved, or updated (augmented) by secure connections over a network. Permission structures, security tokens, and other security measures are employed by EHR and EMR systems to prevent unauthorized access. The methods of the invention encompass the use of such network connections to achieve the retrieval of patient information and the addition of consultation recording files to medical records.

### Part 1B. System Elements

The operations of the disclosed systems comprise various elements, as described below.

Consultations, Users, and Subjects. The systems of the invention may be employed in recording any interaction between two parties. In a primary implementation, the systems are utilized in methods of creating a recording of an interaction wherein the interaction comprises a consultation. The system will be employed by a user during a consultation between the user and a second party, the subject. The user will be the person employing the system, i.e. a person operating a device to carry out the methods of the invention to create and transmit a recording file that captures some or all of the interaction. The user may be a single user or may comprise a group of two or more people jointly using the system or otherwise participating in the interaction, e.g. in concert with the user. The user will be interacting with a subject. The subject may be an individual person, or may comprise a group of two or more persons jointly using the system or working in concert with a subject.

In a primary embodiment, the systems are employed in the medical context. Accordingly, in this context, the interaction will encompass an interaction between a practitioner or provider, and a patient. The practitioner may be, for example, any of a physician, nurse, physician's assistant, health care aid, technician, counselor, dentist or dental technician, emergency personnel, etc. In this context, the subject is a patient, e.g. a person seeking medical care or information. In this context the consultation is a medical visit, e.g. an appointment or consultation, e.g. wherein the health or condition of the patient is evaluated and/or wherein prognosis, diagnosis, medical advice, medical options, or other information is ascertained by the provider and provided to the patient. In a primary implementation the visit is an in-person visit, such as at the doctor's office or house call. Alternatively, the visit may be a teleconference, for example a phone call or video conference.

The systems and methods of the invention may be employed in any interaction between parties, particularly in the context of professional consultation, wherein a provider may be any professional or service personnel, for example, any of an attorney, contractor, mechanic, sales person, etc, for example, any context where a record of the interaction will be useful. In these implementations, the subject may be any of a customer, perspective customer, etc.

It will be understood that with respect to any particular interaction, either party may be the user or the subject. For example, for illustration, in the medical context, the user may be a practitioner, e.g. physician, recording an office visit with a patient. Conversely, the subject may be a patient seeking medical advice and wishing to record the advice of a doctor. As will be described below, the system may be configured for either context, for example, the system may be configured as an app for use by a provider such as physician, and a separate version of the app may be configured for use by patients to record their interactions with a practitioner. In an alternative implementation the app is configured for joint use, for example, configured for operation and inputs by both parties to the interaction.

Consultation Application. A primary element of the systems of the invention is a what will be referred to herein as a “consultation recording and transmission application,” or simply “consultation application.” The consultation application comprises an application (or two or more applications working in combination) utilized to perform or facilitate at least one or more steps of the inventive methods described herein, for example, to receive user inputs which direct the recording of an interaction between the user and another party, and to transmit or facilitate the transmission of a recording file of the interaction. The consultation application advantageously provides the user a convenient means to employ a device, such as a smartphone, to direct recording hardware elements of the device to create a recording of the interaction, and to create a message file augmented with material pertinent to the interaction, which message file may be transmitted to a recipient, for example, the subject of the interaction. The consultation application will be configured to, by the user interface elements of the device, receive commands entered by the user and, in programmed responses to such commands, to direct the operation of hardware elements of the device for the capture and storage of recording data and the transmission of a recording file to selected recipients. An exemplary consultation application, “VISIT REPLAY™” as described in the Examples section of the present disclosure, provides a working example of a consultation application configuration and operations.

In some embodiments, the consultation recording and transmission application comprises communication functions, for example, the application comprises the functions of a communication application, for example an email application, for example, acting as an email client in network connection with an email server or acting as an email server. In this implementation, the functions of the communication application described herein are integral to the consultation application, i.e. a separate communication application is not necessary or not employed. Exemplary functions of the communication application that may be integrated into the consultation application include the creation of message files, the population of message files with data and files (e.g. the recording data) or attached files, the formatting of the message file for transmission over a network, and the transmission of the message to a recipient, such as a communication server (i.e. email server).

Local implementation. In one implementation, the Consultation Application of the invention comprises a local application. A local application comprises an application residing on a device wherein the operations of the application are carried out by the processor of the device, for example, not requiring any external application programs or application for its device-facing functions. In this implementation, the consultation application is loaded onto the device and operates there by the operation of the device's one or more processors. For example, the VISIT REPLAY™ application demonstrated in the Examples section of the present disclosure is an example of a local application.

In this implementation, the function of the consultation application is to receive user commands, direct the creation of data files and recording files, direct the capture of recording data, and direct the transmission of a message comprising the recording data, and optionally, to direct the erasure of data following transmission.

Distributed Application. In another implementation, the consultation application of the invention comprises a distributed application. A distributed application in one embodiment is a program that runs on more than one processor, wherein the two or more processors are connected by a network. In one implementation, the distributed system comprises two separate software programs having a client-server relationship, wherein the front-end or client software operates on the user's device, and the back-end or server software runs on a server, processor, or computer remote from the device and connected to the device by a network.

In the distributed implementation, the front-end software operating on the device processes user inputs, via the input interface hardware elements of the device, and displays outputs for the user, i.e. screens, via the output interface hardware elements of the device. The front-end software may also engage device hardware elements such as microphones and cameras, and may direct the transmission of data captured by these elements to the client-side server.

In one embodiment, the distributed application comprises a first, front-end software program residing and operating on the device and a second, back-end software program or programs operating on a remote system, as described below.

Web Application. In one implementation, the consultation application comprises a web-based application. In this implementation, there is no separate consultation application software residing (stored) on the device. Instead, a web browser application running on the device: provides the operating environment for the device-facing functions of the application; works with the user interface elements, and other hardware elements of the device to receive user inputs and commands and to display system outputs for the user; operates the recording hardware elements of the device to create a recording file; and operates the network elements of the device to transmit data back and forth over a network connection to a back-end processor. The back-end processor, remote from the device, provides processing functions of the web-based application and remote data storage elements store data transmitted to and received by the device. In one embodiment, the web-based consultation application operates as a local browser application in connection with a remote system, as described below.

Consultation Data File. In some embodiments, a primary element of the invention is what will be referred to as a “consultation data file.” The consultation data file refers to the one or more data files which store information about a specific consultation session, which data is used to direct a message and populate a message with content. While the consultation data file is referred to herein in the singular, it is understood that a consultation file may comprise two or more data files which are used to store data concerning a specific consultation session, for example, a doctor's office visit. The consultation data file may be a temporary file. The consultation data file may be encrypted. The consultation data file may comprise a template file comprising structured data fields populated with data specified by user inputs and selections. The consultation data file may be stored locally on the device, remote from the device, or may comprise a distributed file present on two or more memory or storage elements, e.g. one file on the device and another stored remotely. The consultation data file may be stored in a data storage element, i.e. as non-volatile memory. Alternatively, the data file may be stored in working memory, i.e. volatile memory.

In one embodiment, consultation data is stored in the device's local storage using a relational database or like platform for storing structured information, such as SQL, SQLite, ORACLE DATABASE™, etc.

The particular data that is stored in the consultation data file will vary. In most implementations, the data file will comprise one or more recipient addresses to which the recording file is to be transmitted. In one embodiment, the one or more recipient addresses is an email address. The consultation data file may comprise any number of subject data items, such as the subject's name, medical condition, medical record number, symptoms, health status information, insurance information, etc. The consultation data file may comprise any number of links or file references, for example, a reference to a file that is to be included in an email message as an attachment. The consultation data file may comprise data concerning the particular consultation for which it is created, such as the provider's name, the hospital or medical facility name, data specifying the date and time of visit, etc.

Secure Folder. In one implementation, the one or more consultation data files and the recording data file are stored in a secure folder. In one implementation, the secure folder is a temporary folder created upon receipt of the user's command to initiate a new consultation recording session. A secure folder is a folder specifying data that may not be accessed by other applications on the device without granted permissions. In one embodiment, the consultation folder is a temporary folder, for example a directory specified to hold temporary files. In one embodiment, the secure folder is stored in a storage element of the device, e.g. a local drive. In one embodiment, the secure folder is stored in a data storage element remote from the device, i.e. accessed by network connection.

Configured Devices. The scope of the invention encompasses various devices, and their use for performing the operations of the inventive methods described herein. In one aspect, the scope of the invention encompasses what will be referred to herein as “configured devices.” Configured devices comprise devices that are configured, for example, by loading and operation of consultation application elements, to perform one or more functions of the inventive methods. For example, in one embodiment, the configured device stores, by a local data storage element, and operates, by a local processor, a local consultation application. In one embodiment, the configured device comprises a software program comprising the front-end (client) software element of a distributed consultation application. In one embodiment, the configured device comprises a web browser application accessing and/or running a web-based consultation application.

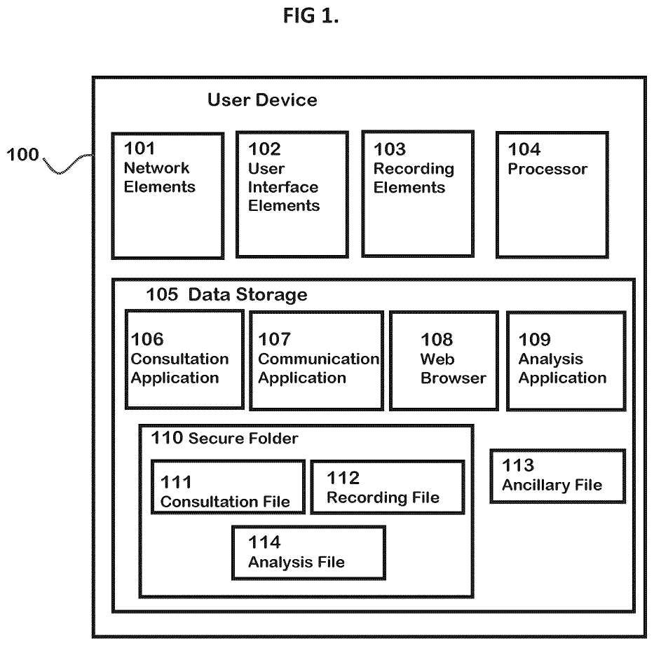

An exemplary configured device of the invention is depicted in FIG. 1. FIG. 1 depicts a device 100 comprising network elements 101, user interface elements 102, recording elements 103, one or more processors 104, and a data storage element 105, wherein the data storage element stores, in some embodiments, a local or front-end consultation application 106, a communication application 107, such as an email application, a web browser application 108, and, optionally, one or more analysis applications 109. The data storage application may further comprise one or more consultation data files 111, a recording data file 112, and one or more analysis files 114, for example, stored in a secure folder 110. The data storage element may comprise one or more ancillary files 113. In various implementations of the invention, certain of the foregoing elements may be omitted from the configured device. For example, in the context of a consultation application comprising a local application, the configured device may not require or comprise a web browser application 108. In the context of a consultation application comprising a web-based application, the configured device may not require or comprise consultation application software 106.

Recording Files. The scope of the invention encompasses the capture of an interaction by recording elements (e.g. microphones and cameras), which data will be referred to herein as “recording data.” The recording data pertinent to a particular consultation is stored in a file that will be referred to herein as a “recording file” or “a file comprising the recording data.” It is understood that recording data captured in an initial recording file may be copied to, exported to, or otherwise duplicated in additional recording files embodying the original recording data. For example, audio recording data may be captured by a device in a raw format such as WAV or PCM data. Following completion of a recording, for example, upon the user's command to terminate a recording, the raw data may be converted to a different file format, such as MP3, M4A, MP4, etc. The data may be further converted for transmission by a communication application, such as in an email attachment file comprising the recording data. In some embodiments, the file is encrypted.

Remote Systems. The scope of the invention, in certain implementations, encompasses the use or operations of what will be referred to herein as a remote system or remote server. The remote system comprises a combination of hardware and software elements that are remote from the device. The remote system may comprise a singular integrated system or a distributed system of networked elements. Depending on the particular implementation of the invention, as described below, the remote system may perform any number of functions. In some implementations, the remote system comprises an EHR or EMR system. In some embodiments, the remote system comprises a secure repository, for example, HIPPA-compliant database, an insurance database, or other repository for long-term storage or archiving of the recording of the interaction.

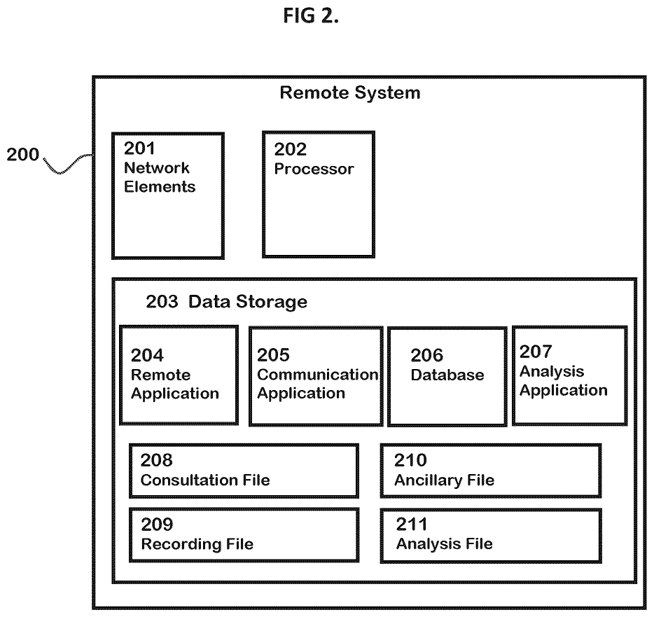

An exemplary remote system of the invention is depicted in FIG. 2. The remote system 200 comprises network elements 201 that enable its connection to a network, and one or more processors 202. The remote system will further comprise one or more data storage elements 203, which store various applications and dat. Applications operating on the remote system may include software programs of a consultation application 204, for example, a back-end module of a distributed consultation application or the primary module of a web-based consultation application. The remote system may comprise a communication application 205, such as a client email application or an email server. The remote system may comprise one or more databases 206, for example, a medical records database. The remote system may comprise one or more analysis applications 207. The remote system may comprise stored files, for example consultation data files 208, recording data files 209, ancillary files 210, and one or more analysis files 211.

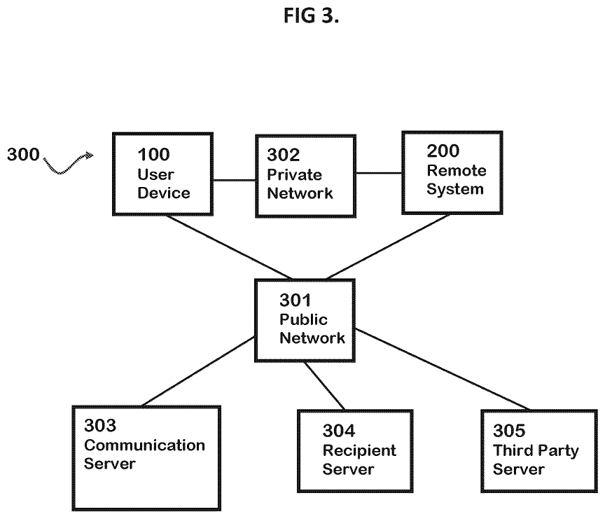

Network Connections of the System. The various elements of the systems described herein may be connected by any number of network connections. FIG. 3 depicts exemplary network connectivity between elements of the system or accessed by the system for certain functions. The device 100 of a user is connected by network connections to a public network 301. By this network connection, the user device may access and communicate with a remote system 200. In some embodiments, the device is connected 100 to the remote system 200 by a private network. By the public network, the device may connect to any of a communication server 303, a recipient server 304, and a third party server 305.

User Guide Material. During the recording of a consultation, the consultation recording and transmission application may be configured to display guide material, for example, written or visual items displayed by the device for the user as a page or series of pages to guide the user's interaction with the subject. The displayed information may comprise any information potentially helpful to the user for the interaction with the subject.

In some implementations, the guide material displayed during the recording session may comprise a script. The script may comprise a series of items to be read aloud or generally followed by the user. In some embodiments, the script can be scrolled or may comprise a series of pages moved to the next page by the user checking a displayed box (e.g. labeled “done” or “next”) or swiping.

In some embodiments, the displayed guide further comprises checkboxes or text fields wherein, by the user interface elements of the device, the user may enter or select items or notes.

In the context of a medical visit, in one embodiment, the script is a permission script read by the practitioner to the patient advising them that a recording of their visit is in progress and confirming that the patient assents to being recorded. The patient may further be asked if they assent to the recording being used for specified purposes, such as for a medical or insurance record, for research, for customer service evaluation, etc. The script may be tailored to the privacy laws and policies of various jurisdictions or institutions (e.g., hospitals), for example, as required by state law.

In the context of a medical visit, the guide information displayed during the recording session may comprise a checklist, for example a list of diagnostic questions or observations. For example, if the record has been specified as a shoulder injury evaluation, the checklist may comprise a list of movements, or visual depictions thereof, to be made by the patient to locate a source of pain (for example, “have patient raise arm laterally, ask if there is pain”). In some embodiments, the displayed guide further comprises checkboxes or text fields wherein by which, by the user interface elements of the device, the user may enter or select items or notes. For example, in the case of the shoulder workup described above, the user may be presented with a menu of checkbox items such as “no pain, minor pain, major pain,” which inputs are saved to the consultation data file. These inputs may be utilized by an analysis application, for example, an analysis application comprising a diagnostic tool, as described below.

In the context of a medical visit, the displayed checklist may comprise displayed information or prompts regarding risks or side effects that the patient should be aware of.

In another embodiment, in the context of a medical visit, the displayed checklist may comprise prompts to ask the patient if they understand the information that has been presented to them and if they have any questions or concerns. For example, the user may be prompted to ask questions such as “do you understand,” “do you have additional questions,” or, “can you relate back to me what I just explained to you.”

Analysis Applications. The recording data may be analyzed by one or more analysis applications, also referred to herein as analysis tools. An analysis application or analysis tool, as used herein, is a computer-implemented tool comprising an application which performs an analysis of one or more aspects of the recording data of a consultation by automated means and creates one or more analysis output files. For illustration, an example of an analysis application is a transcription tool comprising speech recognition functions that analyze the voice data of an audio recording and converts the audio data into a written transcript of the conversation. In this case, the analysis application is a voice-to-text converter and the analysis output file is a transcript.

In one embodiment, the analysis application is applied to the recording file data shortly after the recording is completed. For example, the consultation application may be programmed to apply an analysis application to the recording data immediately after the user enters a command to end the recording. The output of the analysis will be an analysis file comprising the end product of the selected analysis. For example, if the analysis application is a voice-to-text converter, the output will be an analysis file comprising a transcript.

The consultation application may be configured to apply one or more analysis tools to the recording data. In one implementation, the user is presented with or may access a displayed menu and may direct the application of an analysis tool by entering a command. In another embodiment, the consultation application is configured to automatically apply an analysis tool to the recording data following completion of the recording. In one embodiment, the user's command to terminate the recording automatically triggers the application of an analysis tool to the recording data.

In some embodiments, the analysis application is locally stored and operated on the device. In some embodiments, the analysis application is a standalone application. In some embodiments, the analysis application functions are integrated with the consultation application. In some embodiments, the analysis application comprises a plug-in or module that may be applied to analyze the recording data. Upon completion of the analysis, an analysis output file is generated and stored in a data storage element of the device, for example, in a secure folder In other embodiments, the analysis application is remote to the device, i.e. it is stored remote to the device, and operated by processors remote to the device. In one embodiment, the analysis application is accessible as a web-based application, such as a browser application of the device. In one embodiment, upon the user's completion of the recording, the recording data is automatically transmitted via a network connection to a destination wherein it is accessible to a remotely operating analysis application. Following completion of the analysis, an output file summarizing the results of the analysis is transmitted back to the device, or remote system, from which it was sent. Analysis files may be encrypted.

In one implementation, analysis files are provided to the subject of the consultation. For example, in a medical context, the analysis file may comprise a written transcription of the consultation, which is provided to the patient in a message, for example an email message. In this implementation, following completion of a recording, one or more analysis files may be generated by application of one or more analysis applications to the recording data and, at the user's direction, or automatically, the one or more analysis files are included in the message that is sent to the user, for example as embedded content or as attachment files. For example, in the context of a medical visit, an analysis tool comprising a voice-to-text conversion application may be applied to the recording data, the output of which is a file comprising a transcript of the conversation, which file may be attached to an email message which is sent to the patient.

In one implementation, one or more analysis applications are applied to generate analysis outputs that are provided to the user. In this implementation, the system is configured to apply an analysis tool to the recording data of a consultation and to output an analysis file that is transmitted, or otherwise made available to the user (e.g. stored in a database accessible to the user). For example, the analysis file may be provided to the user in a message, e.g. an email, or as displayed material in the consultation application. In this implementation, the analysis tool is employed to provide the user with helpful material, such as a synopsis of the consultation, a list of follow-up action items, a performance evaluation, and other materials that summarize or provide feedback to the user, as described below.

Real-Time Feedback. In some embodiments, the analysis application is applied to the captured recording data in real-time, i.e. the analytic functions of the analysis application are applied to the recording data as it is captured or shortly after it is written to the recording file. In this implementation, analysis application outputs may be displayed for the user on the device as the consultation progresses. This implementation advantageously allows the user to receive feedback on the consultation as it progresses.

Analysis applications. Analysis applications may comprise any number of automated analytical tools known in the art. In one implementation, the analysis tool comprises a language model, for example, a large language model, which has been trained on very large datasets to analyze conversations to assess any number of specified factors. Large language models include Al models such as CHATGPT™, for example, GPT 3.5 and GPT-4.0, BARD™, ERNIE BOT™, and LLAMA™. Language models can be trained to recognize, categorize, and quantify what was discussed during the consultation, by words, phrases, and other aspects of the conversation between the user and subject.

In some embodiments, the analysis tool is applied to the recording data, for example, voice data in an audio file. In other implementations, the recording data, for example, audio data is processed by a first analysis tool comprising a transcription tool, to convert the audio data to a written transcript. One or more additional analysis tools may then be applied to analyze the written transcript of the consultation, rather than analyzing the audio data directly.

Transcription Tool. In one embodiment, the analysis tool is a voice-to-text converter and the analysis output file is a transcript. Exemplary tools for voice-to-text conversion include REV AI™, DRAGON™, OTTER™, and TEMI™ transcription tools.

Translation Tool. In one embodiment, the analysis application is a translation tool. A translation tool analyzes the audio recording, or a written transcript thereof, and converts the conversation from a first language to a second language, creating a written transcription of the conversation in the second language. For example, if the conversation is in English, a translation tool may be used to create a transcript in Mandarin, Spanish, etc. This provides subjects, for example, patients, an ability to review the consultation in their first language when the consultation was performed in a second, less familiar language.

Summary Tool. In one embodiment, the analysis application comprises a language model trained to recognize various items in a medical consultation, such as what topics were discusses, what treatment options were discussed, what risks and benefits were discussed, follow-up items that were discussed, and other features of the consultation. The summary tool detects the elements were discussed in the consultation and provides a document comprising a summary. In one embodiment, the summary is generated for the provider, for example, formatted as a clinic note or like synopsis of the visit using terminology familiar to a medical provider. In one embodiment, the summary is generated for the subject patient, for example, formatted using language that is more readily comprehended by lay persons.

Diagnostic Tools. In one embodiment, the analysis application comprises a diagnostic application, for example, an application configured to analyze the conversation between the provider and the patient in order to formulate a diagnosis, for example by a series of decision tree rules developed by training on large data sets. For example, in the context of a psychiatric or psychological consultations, the subject's speech and language choices may be analyzed by an analysis tool trained on large patient datasets to identify conditions or potential conditions. In other contexts, the symptoms described by the patient during the conversation may be detected and fed into a diagnostic algorithm or like tool to generate a diagnosis.

Provider Performance. In some embodiments, the analysis application is applied to generate an evaluation of the user's performance. For example, in the context of medical visits, there is value in giving the provider feedback on how effectively they communicated to the patient. Such tools can help to train physicians and other providers to be more effective communicators and provide better care. For example, automated tools may be applied to evaluate bedside manner, patient comprehension, and thoroughness of the consultation.

In some embodiments, the analysis application comprises a performance evaluation tool that evaluates one or more performance aspects of the provider-patient interaction. Such tools may comprise language models for example, natural language processing tools (NLPs), trained on large datasets, to analyze factors such as the provider's tone of voice, speed of speaking, word choice, volume of speaking, body language (in the case of video recordings), and the patient's reactions, responses and questions, and other cues to assess qualitative aspects of the interaction In the context of a medical visit, the application of such tools may assist the practitioner user in assessing whether the patient is understanding the complex medical information being imparted. Exemplary emotional assessment tools include COGITO™, GENESYS™, and AFFECTIVA™ products.

For example, analysis tools may be trained to assess performance aspects such as tone, rapport, empathy, bias, how many times the provider interrupted the patient, and for what percentage of the conversation each party was talking, for example “Dr. Smith talked for 84% of the time and the patient talked for 16% of the time.” The analysis output may be a score, for example, a score that is compared to the user's past or recent performances, in order to assess improvement. In this way, the analysis application may help practitioners to improve their communication skills, bedside manner, or other aspects of their communication. Furthermore, by such evaluations, practitioners may be trained to utilize certain keywords or phrases that assist the analysis application in assessing what was discussed.

In one embodiment, the analysis tool assesses the language of the subject, for example assessing whether the subject seems engaged, confused, frustrated, or confident. In one embodiment, the analysis tool assesses whether the subject is comprehending the information being communicated and indicates to the user how well the material is being absorbed by the subject.

In one embodiment, the analysis application comprises a language model is trained to recognize outcome-determinative words, phrases, or patterns. Factors that are indicative of outcome, such as the number of times a patient is asked if they understand or the number of questions a patient asks, can be assessed to provide nomogram-type data that estimates positive outcome probability (e.g., health outcomes, legal outcomes, patient satisfaction outcomes, etc.) based on detected language in the consultation.

In one embodiment, the analysis application comprises a language model is trained to assess the school grade reading level of the user and the subject (provider and patient), or a like metric for assessing educational level or technical sophistication. The analysis tool determines if there is a mismatch between the provider and patient that will potentially lead to a lack of understanding, for example, a provider speaking to a patient having a 5th grade reading level using Post-Doc level language and phraseology.

In one embodiment, the analysis tool analyzes one or more aspects of the conversation between the user and the subject and provides feedback to the user in real-time. In another embodiment, the analysis application is applied post-hoc to the recording and a summary is provided to the user, for example as an emailed report.

Medical Billing. In one embodiment, the analysis application comprises a medical billing analysis tool which analyzes the interaction and outputs an analysis file listing medical billing items that were detected. The analysis application may comprise an NLP or other language processing model that has been trained to recognize various words or phrases associated with billable items. For example, a language processing tool may be trained to recognize discussion of various medical topics and the time that was spent discussing each topic (for example, “4.5 minutes was spent discussing risks of the proposed treatment”) and other billable features of the interaction. Billable features may includes words from the discussion (diagnoses, topics discussed) that indicate the complexity of the discussion in order to assess the appropriate level of billing merited by the discussion. A list of detected billable items may be generated as the output of the analysis, and the file comprising this list may be displayed for the provider, transmitted to the provider, or may be transmitted to a billing department or service.

Checklist Tool. In one embodiment, in the context of real-time analysis, the analysis application comprises a checklist tool. The checklist tool may comprise a language processing model trained to recognize words and phrases associated with certain topics pertinent to a specific type of consultation. In this implementation, the analysis tool is applied to the recording data as it is captured and the analysis tool detects whether certain items have been discussed, and discussed/not discussed status is displayed for the user on a display of the consultation capture and transmission application in order to apprise the user of what remains to be covered. For example, in the context of a medical visit, the user may specify that the consultation is a radiotherapy follow up visit wherein a checklist of items pertinent to such visit is displayed for the user. The analysis application, by language models trained to recognize items discussed in a radiotherapy follow-up consultation, will access the recording data as it is captured and when an item on the checklist is discussed, this will be indicated to the user, for example by displaying a green checkmark, removing the item from the checklist, or some other indicia that the item has been covered. In this way, the user is guided to cover all the important items that should be covered in such a visit.

### Part II. Methods and Operations of the System

In the sections that follow, the methods and operations of the system will be described and various exemplary embodiments will be set forth to illustrate various implementations of the methods, devices, and systems disclosed herein. It will be understood the methods and processes presented herein are exemplary only and the general operation of the system is not limited to a specific order of steps or the requirement that all steps be performed. The illustrative methods presented herein may be practiced in alternative implementations with variations on the order of steps, the omission of recited steps, the addition of additional steps, and other variations.

General Method of the Invention. Methods and processes of the invention performed by the system will be described next. In one implementation, the scope of the invention encompasses a general method as follows:

A method of creating and transmitting a recording of a consultation between a user of a device and a subject, comprising the following steps:
the device is engaged by the user, wherein
the device is a networked device;
the device comprises recording hardware elements; and
wherein the device further comprises or has network connections to:
   a data storage element;
   a communication application; and
   a consultation capture and transmission application;
by user interface elements of the device, the user enters a command directing the consultation capture and transmission application to initiate a recording session;
one or more consultation data files is created and stored in the data storage element;
by user interface elements of the device, the user inputs or selects consultation data, the consultation data comprising one or more recipient addresses, which consultation data is written to the one or more consultation data files;
by user interface elements of the device, the user enters a command directing the consultation recording and transmission application to initiate a recording;
a recording file is created in the data storage element;
the consultation recording and transmission application directs the recording hardware elements of the device to capture recording data comprising audio or audiovisual data, which recording data captured by the hardware elements is written to a recording data file;
by user interface elements of the device, the user enters a command directing the consultation capture and transmission application to terminate the recording;
by user interface elements of the device, the user enters a command directing the consultation capture and transmission application to initiate a transmission of the consultation recording;
the communication application of the device is engaged and directed to generate a new message file;
the communication application creates a message file addressed to the one or more recipient addresses and populated with data from the consultation data file and a file comprising the recording data, or a link thereto; and/or a written transcript of the interaction;
the user enters a command directing the communication application to send the message; and
the communication application, via network connection, transmits the message to the one or more recipient addresses.

The foregoing method may be implemented in various ways. For example, as set forth below, the method may be implemented on a device by the operations of a local consultation application. In another embodiment, the method may be implemented by use of a consultation application comprising a distributed application residing in part on the device and in part on a remote system, such as an EMR or EHR system.

Upon the user's entry of a command to initiate a new consultation recording session, one or more consultation data files is created and stored in the data storage element. In one embodiment, the data storage element is on the device. In another embodiment, the data storage element is a remote data storage device in network connection with the device.

By one or more displayed screens on which the user may make inputs or selections, the consultation application may be utilized to collect various information pertinent to the session, which data will be stored in the one or more consultation data files. For example, the consultation application may be configured to display a screen or series of screens that prompt the user to input or select various information about the consultation session. In some embodiments, the information is entered by the user. In some embodiments, based on user selections, the information is retrieved from a remote source in network connection with the device, such as a remote server or remote system, for example, an EMR or EHR system. For example, in the context of a doctor visit, the practitioner user may be prompted to enter or select the patient subject's name, date of birth, medical identifier numbers, email address, and other information relevant to the visit. In some embodiments, the type of visit is input or selected (for example, from a menu of visit types, e.g. “post-operative follow-up evaluation”) Geolocation and timestamp data may be generated by suitable elements operating on the device and stored in the data file. The data specified by the inputs or selections will be stored in the consultation data file, for example as data strings in designated fields of a data file. The data strings may comprise references or links to additional files, for example, image files, PDFs, web pages and other items wherein the reference or link identifies the files and their location. The prompts may be presented in a series of nested menus.

In the context of a medical visit, the practitioner user may input observed information (“Feverish, temperature 101° F.”) or reported by the patient (“Ms. Smith says her shin hurts”), for example, in a notes field of a displayed screen, by a displayed or tangible keyboard, wherein the inputs are written to the consultation data file. The inputs and selections may include ancillary files, as described below, specified for later transmission in the message, as set forth below.

Recording. When ready to record the session, the user initiates a recording by entering a command, such as selecting a displayed button, which directs the consultation application to initiate the recording. Upon receipt of such initiation command, the consultation application directs the creation of a recording file in the data storage element. Alternatively, the recording file may be created at an earlier time point. In some implementations, the recording data file is stored in a secure folder stored in a data storage element of the device. In one embodiment, the recording data file is created locally in a data storage element of the device. In another embodiment, the recording data file is created in a remote data storage element in network connection with the device.

Upon receipt of the initiation command, the consultation application, for example by its APIs with the drivers and control elements of the recording hardware elements of the device, engages one or more recording elements, for example a microphone and/or camera in video mode and directs the engaged element to commence capturing data. In one embodiment, the captured data is audio data captured by a microphone. In on embodiment, the captured data is both audio data captured by a microphone and video data captured by a camera of the device (or a peripheral camera in connection with the device).

In the context of a medical visit, in some instances, an audio recording is preferred to preserve patient privacy, as a patient may not be comfortable having their body filmed. In other instances, due to the visual nature of the patient's affliction (for example a rash or orthopedic assessment), it may be preferred to create an audiovisual recording of the session in order to capture diagnostic visual information.

Recording data captured by the microphone and/or camera of the device is written to the recording file. In those embodiments wherein the recording file is stored locally, the recording data is stored in a file in a data storage element of the device. In those embodiments wherein the recording file is stored on a remote system, the recording data will be transmitted from the device to the remote system by a network connection. In a primary embodiment, the transmitted recording data is encrypted and/or is sent over a secure network connection

During the course of the recording, the user may be presented with guide materials displayed on the display of the device. During the course of the recording, the user may be presented with a notes field, checkboxes, or other elements wherein data about the consultation may be entered or selected by the user, and wherein any such inputs are written to the consultation data file.

After the consultation is completed, the user then enters a command for the consultation capture and transmission application to terminate the recording, for example, by selecting a displayed “stop” button. In response, the recording data capture functions are terminated and the file comprising the consultation recording data is completed. In one embodiment, the recording data file is converted from a raw format to a compressed format following completion of the recording.

Following termination of the recording, the user optionally may be presented with displayed screens that enable further inputs or selections. Notes fields may be displayed for entry of additional notes about the interactions, and checkboxes, menus or like items may be displayed for selection of additional information. The user may specify files or other materials for transmission to the subject. These inputs and selections are written to the consultation data file. For example, in the context of a medical visit, based on the topics that were discussed, the user may select pertinent material to be sent to the subject. For example, if the administration of a particular intervention was discussed, the provider user may select files comprising background material about the intervention, such as educational material or instructions to be followed by the subject patient.

Creation of Message File. When the user is ready to transmit a file comprising the recording data and any additional material, the user enters a command directing the consultation application to create a message. In one embodiment, the message is created by a separate communication application at the direction of the consultation application. In a primary embodiment, the communication application is an email application and the message file is an email message. In one embodiment, the communication application is a local application. In one embodiment, the communication application is a distributed application, residing in part on the device and in part on a remote server in network connection with the device. In one implementation, the communication application comprises a web-based application, operated through a web browser application on the device and not requiring a separate communication application.

In one embodiment, the consultation application and communication application are integrated such that the consultation application has message creation and transmission functions, for example, email client or server functions. For convenience, the description herein will refer to the use of a separate communication application, however, it is understood that in some implementations, the communication application functions are integrated to the consultation application and no separate communication application is necessary.

The message file is created by the communication application. Exemplary email message file formats include HTML, plain text, .eml, .msg, .pst. and other formats known in the art and will depend on the choice of email application selected.

The message file may be based on a template, comprising a file that provides a structured arrangement of text and/or graphics in the message. In the context of an email file, the template will comprise an email template, for example comprising addressee information and a title in the header and a text body, and default attachments. The template may comprise a default template, a template selected by the user, or a template automatically selected by a rules-based process based on previous inputs made by the user. For example, if the user has specified that the consultation is a post-operative follow up visit, a suitable template pertinent to such visit may be utilized automatically based on that selection. The email file will be populated by information from the consultation data file(s). For example, items such as the recipient email address, a subject's name, and other information present in the consultation data file may populate the email template. If the user has specified files to be included in the message, the references to such files in the consultation data file will direct their retrieval and inclusion in the message, for example, as embedded material or as attachments.

In some implementations, previous inputs by the user will, by a rules-based operation, automatically direct the inclusion of specific ancillary materials (comprising text or files) in the message. For example, in the context of a medical visit, if the practitioner user inputs that the visit concerns a certain type of disease, condition, or intervention, documents or links to documents about that disease, condition, or intervention may be automatically specified for inclusion in the transmission, and will be retrieved and embedded or attached to the message.

In the context of a medical visit, the ancillary material may include documents relating to the patient's care. For example, the practitioner user may specify that a patient subject that has undergone a surgical procedure, and the consultation application will be configured to include a document on how to clean and care for a healing surgical incision in the message. The ancillary material may include documents relating to potential risks or side effects of a proposed intervention the patient is considering, as well as journal articles, clinical trial data, and other information. Ancillary materials may comprise text which is included in the body of the message. Files comprising the ancillary materials may be stored in the data storage of the device or may be stored in a remote data storage element, for example, on a remote system or third party server in network connection with the device.

A primary function of the message is to convey a file comprising the recording data, or access thereto, to one or more recipients. Accordingly, by the command to create a message, the message file that is created will contain, either embedded or as an attached file, a copy of the recording data. In some embodiments, the file comprising the recording data is encrypted prior to its inclusion in the message. In an alternative implementation, for example, in the context of a remote system, wherein the recording data is stored remotely from the device, such as in a database (e.g. a database integral to an EMR or EHR system), the message does not include a file comprising the recording data, but instead includes a link to a file comprising the recording data. As known in the art, a link is a reference, such as a hyperlink, HTML link, or URL that accesses a file stored in a location specified by the link. The link may comprise a link to a password protected or otherwise secure database or storage wherein a file comprising the recording data may be accessed, for example by password, security token, two-factor authentication, or other security scheme to prevent unauthorized access.

In an alternative implementation, rather than sending a file comprising the recording data or a link, a transcript of the consultation is sent to the selected recipients. Transcripts may be generated by voice-to-text conversion tools, as discussed below. In one embodiment, the transcript is encrypted.

In some implementations, the consultation data file(s) and the file comprising the recording data are stored on the device. In some embodiments, the consultation data file(s) and the file comprising the recording data are stored in a secure folder. In such implementation, the system is configured such that the communication application has permissions to access the secure folder to read consultation data file contents and write the data therein to a message file, and to copy the recording data for transmission as an embedded or attached file, for example, in an encrypted form. Permission structures may be granted on a default basis, or at each instance by presenting the user with a popup or like message requesting permission, for example, in some embodiments requiring an authentication step.

In one embodiment, upon the user's command to initiate a message, the focus of the device is switched to the communication application, wherein the communication application displays the draft message for the user. The user may make additional edits to the message here, and may specify the inclusion or attachment of additional files.

In some embodiments, the message is encrypted. In some embodiments, files transmitted with the message (e.g., as attachments) such as recording data files or transcripts thereof, are encrypted. In some embodiments, the message email is sent to a destination email server over a secure network connection.

When the user is ready, the user enters a command for the communication application to transmit the message. In the context of an email, the communication application may comprise an email client and the email message may be sent from the device to an email server, for example, by a public network. The email server may then, by standard SMTP protocols, transmit the message file to the one or more recipient email servers, from which it may be delivered to the recipient by their client email program. For example, referring to FIG. 1 and FIG. 3, the email may be transmitted by communication application 107 from device 100 via a public network 301 (e.g. the internet), to a communication server 303 comprising an email server, and from there, may be routed via public network 301 to a recipient server 304, comprising a destination email server.

In an alternative implementation, upon the user's entry of a command to create a message, the message is created, populated with data from the consultation data file and the recording data, and is sent automatically by the communication application without the need for the user to enter a send command.

In the context of a medical visit, the transmission of the message will typically be to the subject patient, who may benefit from the ability to replay the medical visit so as to better absorb the details of the consultation. The inclusion of ancillary materials may further aid the subject in interpreting the consultation, in making informed medical decisions, and in compliance with medical instructions. The provider user may also be a recipient of the message, for medical records purposes or for memorializing the visit for future reference or to offset malpractice liability.

In one embodiment, the message is transmitted to a medical records system or other repository for storage, for example to a HIPPA-compliant database or repository of an EMR or EHR system. In one embodiment, the message is an email message and comprises information in the header or body which enables a recipient remote system to identify the message as privileged to access the remote system and to recognize a suitable destination for the message, such as a medical record of the patient. For example, the email header may comprise reference numbers or identifiers that specify a suitable storage destination. In some embodiments the message file is automatically CC'd to the user provider or a remote system, for example, an EMR or EHR system. In some implementations, the message file is formatted for transmission to a database, for example, a secure database, for example, a HIPPA compliant database. In some implementations, the database is integral to an EMR or EHR system. The message file may comprise tokens, passwords, EMR/EHR links, or other elements that enable the message to be received by the destination database.

In some embodiments, multiple transmissions may be performed. For example, in one embodiment, a first transmission of a file comprising the recording data to a remote system is performed, wherein the file comprising the recording data is transmitted to a destination comprising a remote system and is stored in a data storage deice of the remote system, for example, in a database. A second transmission of a file comprising the recording data, or a link thereto, may be made to the recipient. Transmission of a link may be preferred for security reasons, requiring the recipient to enter a password or perform an authentication step before they may access the file comprising the recording data from the remote system for playback or download.

In some embodiments, two or more separate message files are generated with differing information and differing destinations. For example, in one embodiment, the user's command to initiate a message results in the creation of a first message file comprising an email message file addressed to the subject and other recipients (e.g. optionally addressed to the user as a CC); and a second message formatted for transmission to a database, e.g. an EMR or EHR system that will receive the recording, any ancillary data or material, and will store it in the subject's medical record.

Following transmission of the one or more message, the disposition of the original consultation data file(s) and recording file can vary. In one embodiment, the consultation data file and/or recording file are stored, for example stored in or written into a database. In one embodiment, the recording data is copied to a database and the original recording file is erased. In one embodiment, both the consultation data file and files comprising the recording data are erased. The options for disposition of these files is illustrated in the exemplary embodiments that follow.

Analysis applications. Analysis applications may be applied to analyze one or more aspects of the recording data. In one embodiment, the user entering a command to stop the recording will direct the system to apply one or more analysis applications to the recording data. In one implementation, the analysis application comprises a voice-to-text conversion tool which generates a transcript of the consultation. In one embodiment the transcript file is included in the message, for example embedded or as an attachment. In one embodiment, one or more additional analysis tools are applied to the transcript file.

In one embodiment, the system is configured to automatically apply one or more analysis tools, for example, comprising a performance analysis tool, to the recording data (or to a transcript thereof). The output of this analysis application may be displayed for the user on a the device, or may be stored as a file that is accessible to the user or which is transmitted to the user in a message, for example, as an email attachment. The analysis tool may be applied to the recording data in real-time, or may be applied post-hoc, following completion of the recording, for example, in response to the user entering a command to terminate the recording.

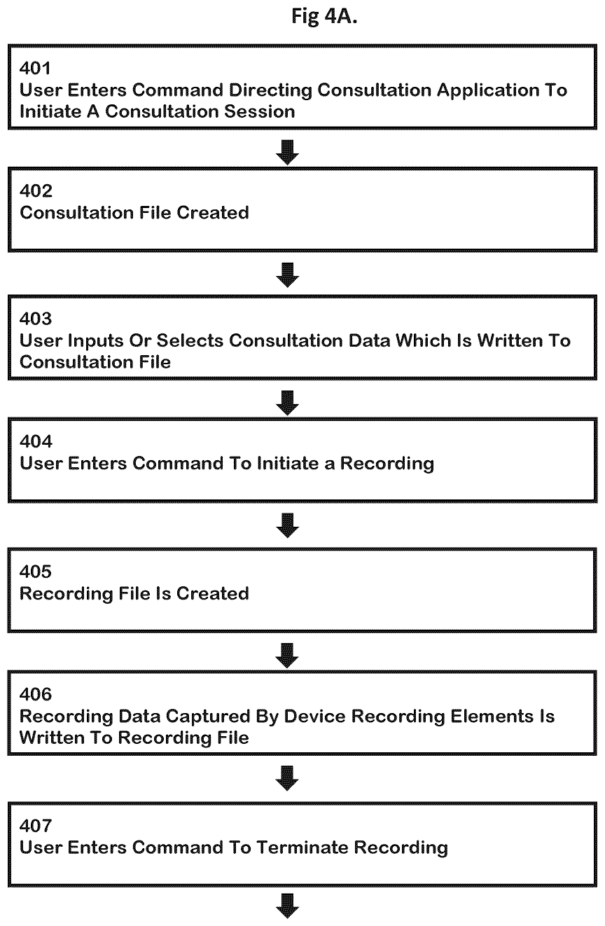

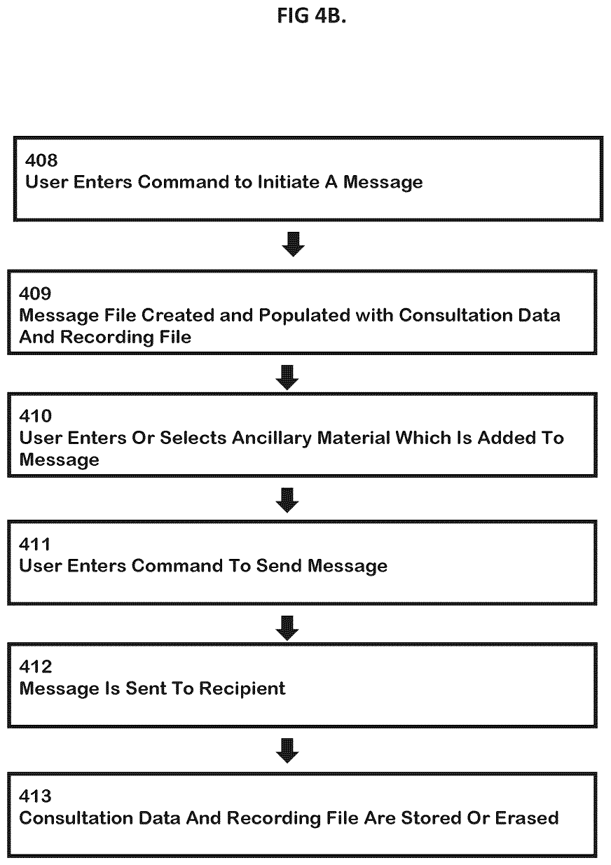

A exemplary process of the foregoing general method is depicted in the flow chart of FIGS. 4A, 4B, and 4C.

Local Application. In one embodiment of the foregoing general method, the scope of the invention encompasses a method of capturing a recording of a consultation and transmitting the recording to one or more recipients by use of a consultation application comprising a local application, i.e., residing on the user's device. In this implementation, the consultation application operates entirely on the user's device.

In one embodiment of the local application implementation, referring to FIG. 1, FIG. 2, and FIG. 3, all data is stored on the device 100, in the one or more data storage devices 105 thereof. The consultation data file 111, recording file 112, and any other ancillary files 113, are stored locally on the device. In one embodiment, the consultation data file and recording file are stored in a secure folder 110.

In the local application implementation, analysis applications may be stored and operate on the device 109. Alternatively, analysis applications may be accessed by network connection from a remote third party server 305, wherein analysis application output files 114 are transmitted to the device and stored in the data storage element 105.

In the local application implementation, the communication application 107 may be a separate application, or may be integral to the consultation application 106, may comprise a local communication application, a client application of a distributed system in network connection with a remote server, or a web-based application. In a primary implementation, the communication application is an email application. In one embodiment, the communication application is a client VPN application.

For data security, it will be advantageous to the user that no confidential data is stored on the device. Accordingly, in one embodiment of the local application implementation, the consultation data file and recording files are erased following their transmission in the message. This advantageously removes potentially sensitive information, for examples, patent medical information, from the user's device.

In one embodiment, the erasure is automatically performed at the direction of the consultation application following transmission, for example, in response to a signal from the communication application that the message has been sent (or received by the one or more recipients). In another implementation, following transmission of the message by the communication application, the consultation application presents the user with a menu, popup, or other displayed item enabling the user to elect erasure. If the user enters a command to erase, the consultation application directs the erasure of the consultation data file and recording file. For example, the user may be presented with a pop-up display such as “Erase Data for this Session?” Wherein, if the user selects “erase” the consultation file is erased. As set forth in an exemplary embodiment below, the erasure may be automatically initiated by the user's election to return to a home screen.

In some embodiments, in addition to erasure of the original consultation data files and recording file, the sent message, e.g., email, is automatically erased from the communication application's sent messages, e.g., sent email folder, for increased security.

Erasure may be performed various ways. In those embodiments where the consultation data file and recording file are stored in a secure folder, and the secure folder is a temporary folder, the entire folder may be erased. Alternatively, the individual consultation data files and recording file may be temporary files that are erased.

Erasure may be by any suitable method, for example, data write-over techniques known in the art, for example, DOD 5220.22-M 3 & 7 Pass, NIST 800-88 standard.

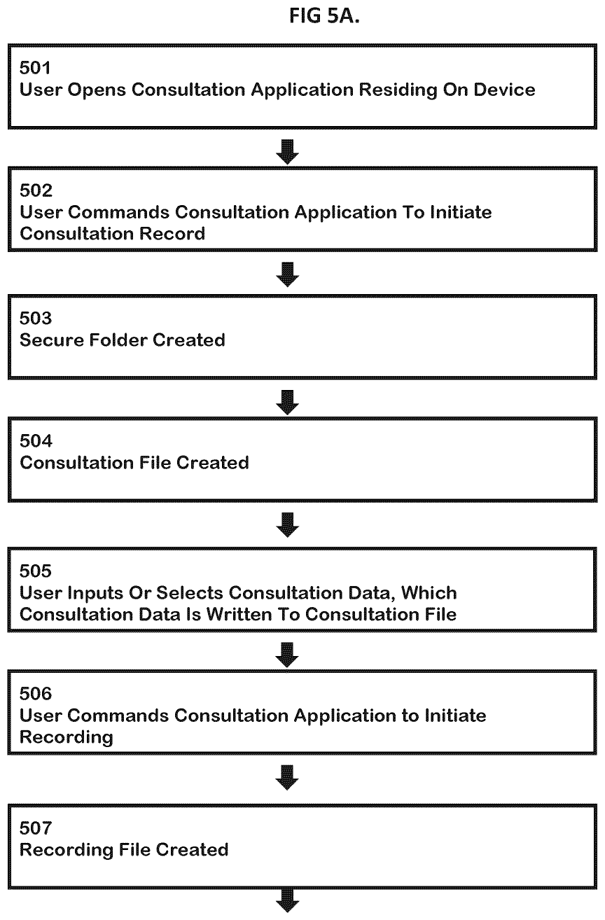

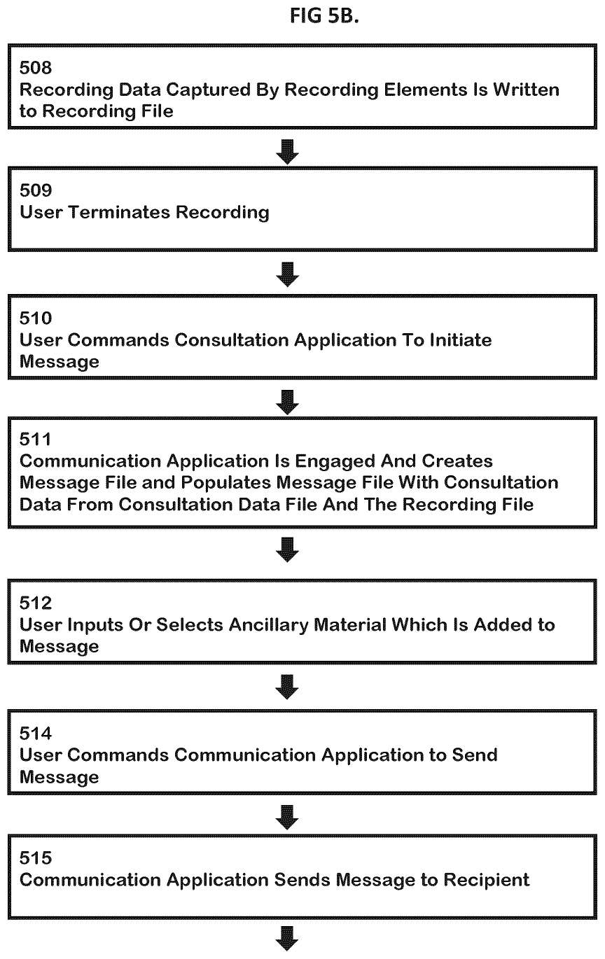

An exemplary embodiment of the local application implementation is depicted in FIGS. 5A, 5B, and 5C. The local application implementation is also illustrated by the VISIT REPLAY™ app demonstrated in the Examples.

Remote System Implementation. In one implementation of the general method, the device operates in coordination with a remote system. In this implementation, the consultation capture and transmission application functions on the user's device as an interface with a remote system. In this implementation, the consultation application comprises a distributed application or a web-based application.

In one embodiment, referring to FIG. 1, FIG. 2 and FIG. 3, a consultation application 106 comprising a client application is stored and operates on the device 100 and coordinates, by network connection via private 302 or public network 301, with a server consultation application 204 of a remote system 200. In another embodiment, no consultation application resides on the device, and the consultation application is operated locally as a web-based application by use of a web browser application 108 operating on the device.

In remote system implementations, the consultation data file(s) 208 and recording file 209 are stored on a data storage device 203 of the remote system. Likewise, ancillary files 210, are stored on a data storage device 203 of the remote system. Analysis applications may reside on the device 109, may be accessed from a third party server 305 by network connection, or may comprise analysis applications 207 present in the data storage 203 of the remote system 200. In this implementation, analysis application output files will be stored in the data storage of the remote system.

In this implementation, the communication application 205 typically operates on the remote system, or the functions thereof are otherwise integral to the remote system 200. Messages comprising consultation data (e.g. patient information or notes about the consultation session), a file comprising the recording data, or a link thereto, any ancillary files selected, and any analysis files included, are created on the remote system and transmitted therefrom. In a primary implementation, the consultation data, the file comprising the recording data, and any analysis files are encrypted prior to transmission.

Following transmission of the message, in a primary embodiment, data from the consultation file 208, and files comprising the recording data 209, and any analysis files 211, may be erased, or may be retained on remote system 200 for long-term data storage, for example, in a database 206.

The remote system implementation of the general method is highly amenable to uses in the medical context, for example, wherein the remote system is a medical record systems. In this implementation, the device functions mainly as a means for receiving user commands, capturing recording data from the consultation and transmitting it to the remote system. No long-term storage of sensitive medical data on the device occurs in this implementation, and the recording is stored remotely and securely as a medical record, which may be played back by the recipient or downloaded.

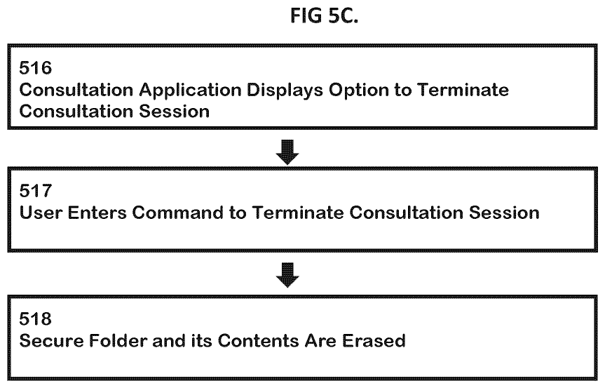

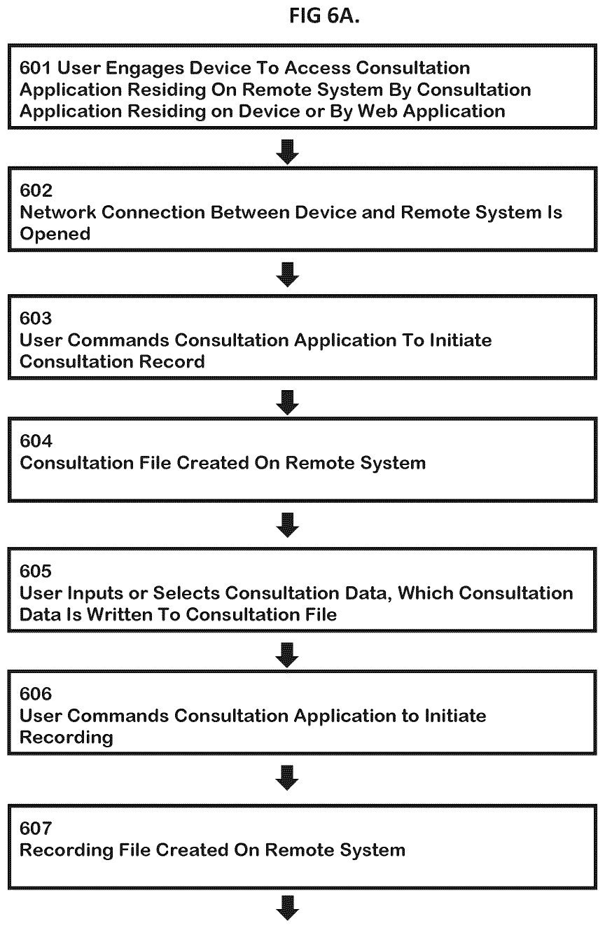

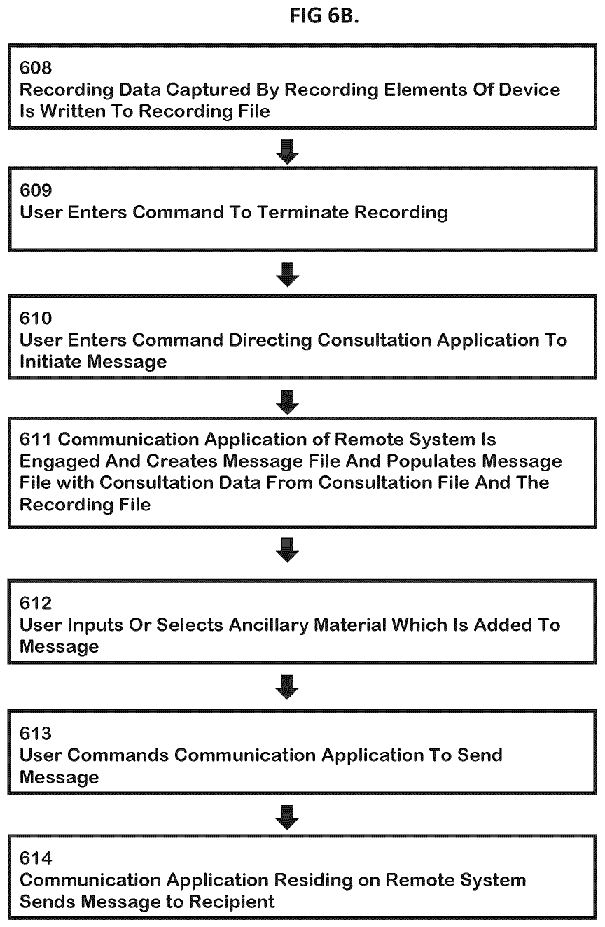

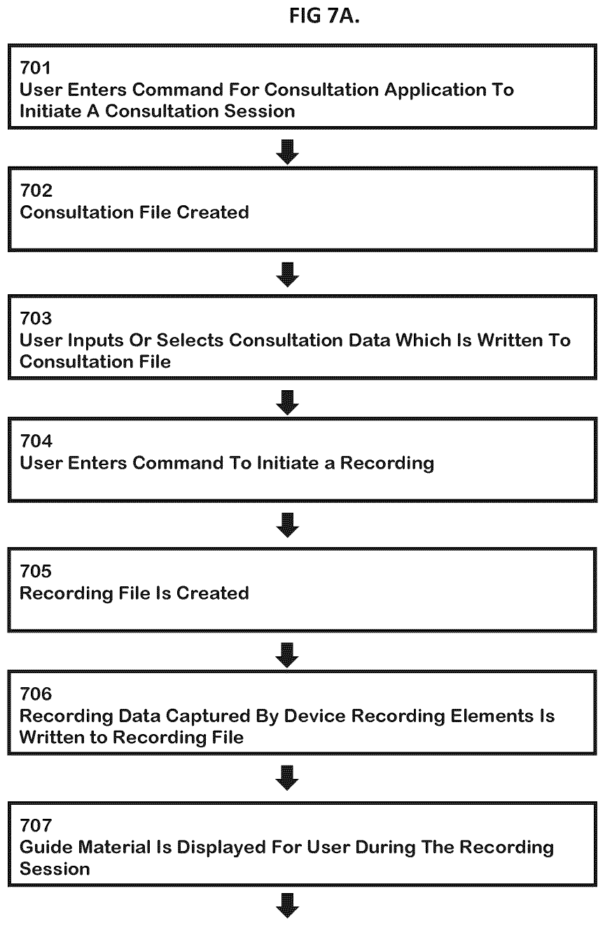

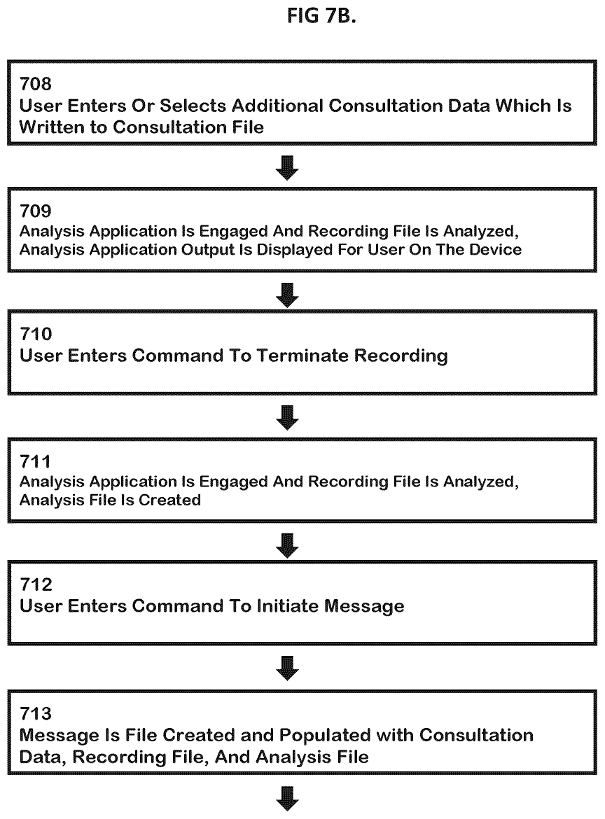

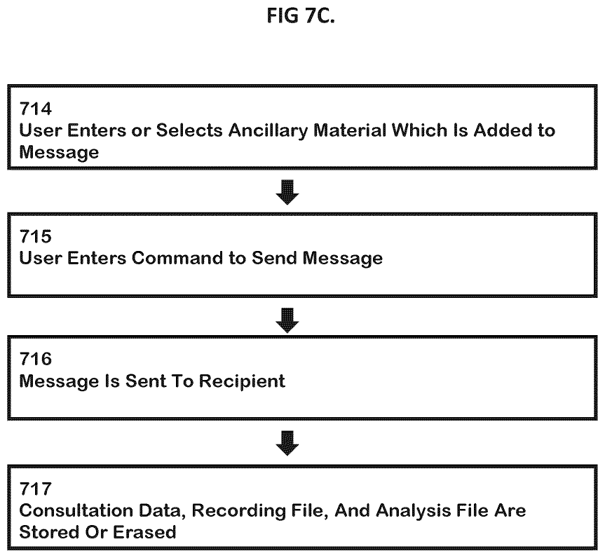

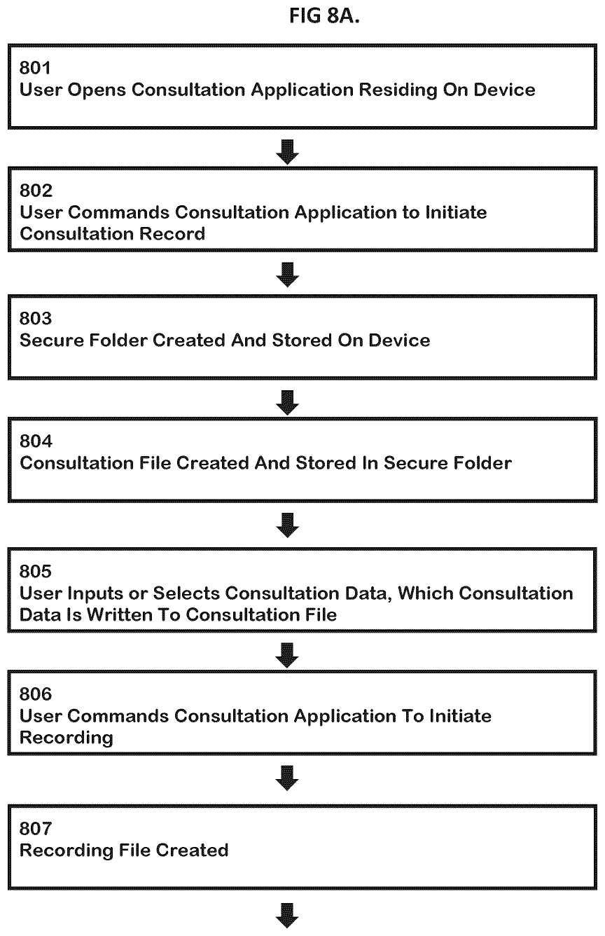

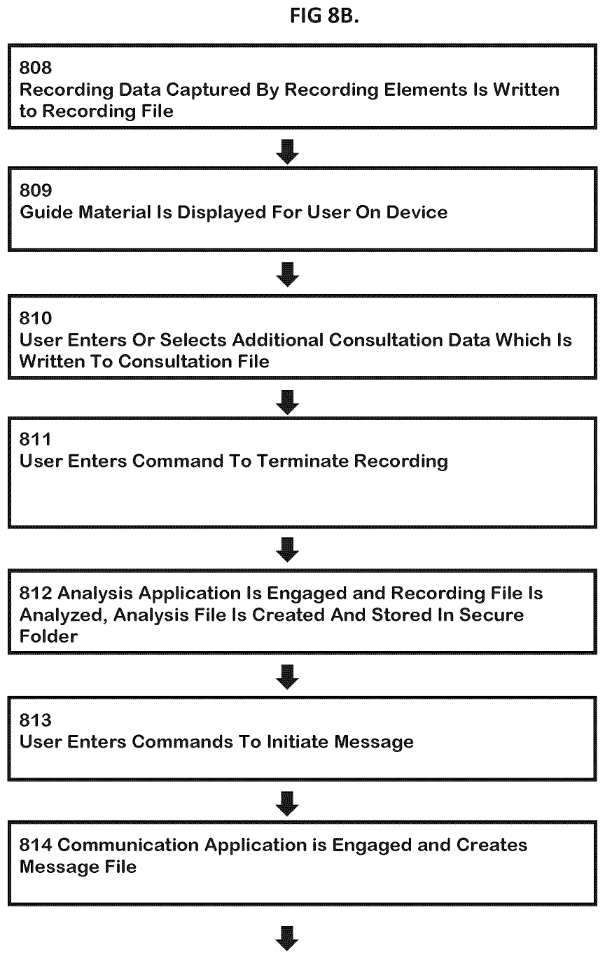

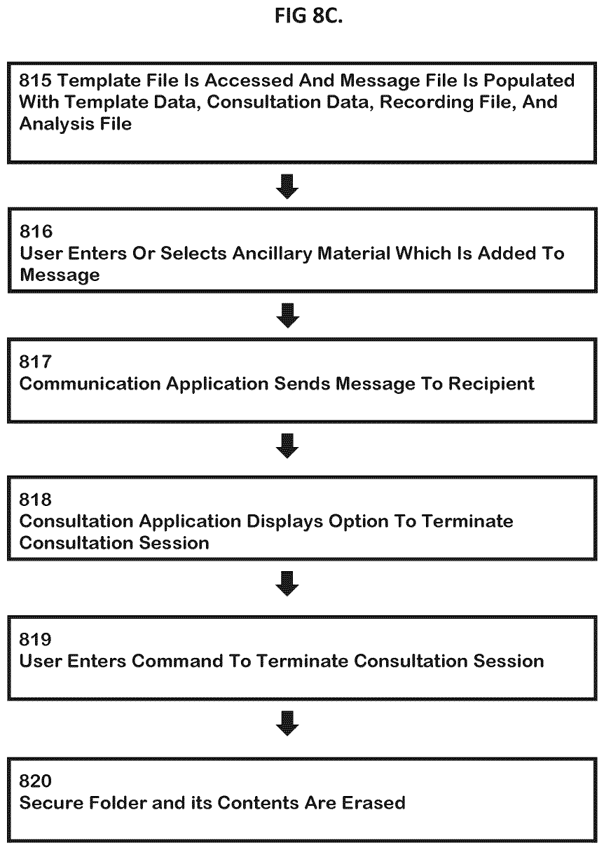

An exemplary embodiment of the remote system implementation is described in FIGS. 6A and 6B.

Medical Context Implementations. Various implementations of the invention are carried out in the context of a medical consultation. Exemplary features are provided for illustration. Upon the user's engagement of the consultation recording and transmission application, an authentication page or screen may be displayed at this point with inputs for password, face recognition etc. The physician may input or confirm his or her identity, and may also select or input the identity of other people present for the visit (e.g. physicians' assistant, nurse, or other person present).

The physician may then be presented with a menu or other display where options may be selected. For example, the physician may input her or his location (e.g. which hospital, clinic, or medical organization she or he is working under for the day). Office data may be ascertained by geolocation functions on or accessible to the device, e.g., smartphone. The patient's name may be input or selected from a menu or search function, or another identifier such as patient ID number may be used. Patient gender may be specified. The type of visit may be input or selected (e.g. from a menu or search function), for example, “oncology consultation” or “UTI follow up visit.”

Each selection or input may dictate which page, menu, fields, data, or screen is displayed next. For example, if “potential fracture” is input or selected for reason for visit, then a menu of potential fracture locations (e.g., forearm, upper arm, leg, rib, etc) may be displayed for selection next.

Each selection or input may result in the app accessing remote data, for example from a database such as an Electronic Medical Records database. Links or tabs may be displayed for the physician in order to view or bring up additional data. For example, input of patient's name may retrieve and display data on the patients' age, health status, last visit date, etc. Retrieved data is stored or referenced (e.g. by link) in the consultation data file.

In some cases, a patient data template is prepared in advance of the visit, for example, prepared by administrative support staff or generated in an automated step by tying in to scheduling software so the physician is able to quickly retrieve patient data without needing to make substantial inputs.

In one embodiment, the patient is asked for their contact information so that the recording may be shared with them. In a primary embodiment, the patient's email address is input so a file comprising the recording data can be sent to them following the visit. Alternatively, a phone number or other contact information may be used.

Upon greeting the patient, the physician may request permission or otherwise obtain informed consent to record the visit. The physician may be presented with script to read which outlines the legal basis for the recording, the patient's rights thereunder, including the right to deny permission to record. There may be authentication tools displayed or provided to verify the patient's assent. For example, the patient may be asked after the initiation of the recording to state their name and that they agree to the creation of the recording. The patient could be presented with a signature block to sign or required to scan a digit or their face to authenticate their consent. In one embodiment, the system can serve as a means of memorializing consent and/or assent (e.g. clinical trials companies can use the app to provide better “informed consent” to study subjects by including study, or drug information in the outgoing email, while at the same time saving the recorded discussion as a recorded proof that certain items, e.g. risks and side effects of a particular intervention or drug were discussed with the patient, and that they understood and/or agreed.

After consent is received, the physician may initiate the recording by engaging a control. For example, a displayed “start” button could be used to initiate an audio recording. Accessing a video camera function and engaging the start record button or like control could be used to initiate a video recording. The controls, e.g. displayed controls may further encompass pause functions and a stop button.

Conveniently, in smartphone implementations wherein an audio recording is being made, the phone or like device can be left in the vicinity and in a recording mode without affecting or interfering with the visit. Alternatively, the physician may tailor his or her actions to augment the recording, for example, providing narrative to note what is happening during the visit, for example, “I am palpitating the lesion on Mr. Gurbick's forehead now.” In the case of a video file being recorded the device will be positioned to capture the interaction, for example, by the aid of a tripod.

If the recording is to be paused, for example, to exclude material from the recording, a “pause” control may be engaged. When the visit is complete, the physician can engage a “stop” control to end the recording and commence the next steps in processing of the recording data created in the recording process.

In one embodiment, during the visit, prompts are displayed for the provider. The App can include a list of specific prompts that the user says aloud, and which the patient has to audibly respond to, such as “do you understand”, “do you have additional questions”, or, “can you relate back to me what I just explained to you,” etc, all of which are preserved in the audio recording to guard against future disputes, etc. Audio tones or sounds can be used to remind the provider to say required prompts.

In one implementation, the system is configured so that the message may be augmented with additional information relevant to the visit. For example, this can provide additional medical guidance to the patient or act as a reminder to reinforce directions given to the patient during the visit. In one implementation, when the recording is complete, the user is presented with a screen giving them the option to add additional material to the email. The user may be presented with a menu that enables them to select additional information, in the form of web address links and/or documents to add to the\email. In one embodiment, the selection of links and/or attachments that is presented is selected based on the earlier-entered patient data, i.e. the items that are presented for potential inclusion are items that are relevant to the patient's condition. For example, if the subject is an ovarian cancer patient, the selection of links and/or attachments in the menu could be links and files such as links to relevant websites about ovarian cancer and PDFs of papers describing ovarian cancer clinical trials. Ancillary material files may be stored locally on the device, or accessed remotely via links.

In one version of the app, the process is aided by the use of message templates, i.e. email templates. The user may create a template that contains a selected suite of additional information relevant to a particular condition. For example, if the provider tends to see many subjects having a particular condition, the provider can build a template email that is formatted with the additional material included, including embedded links and/or documents attached. For example, an orthopedic surgeon that operates on knees, wrists, and shoulders might create separate templates for each condition with information about post-operative care and physical therapy that is relevant to each. Prior to initiating the recording, at the patient data input stage, the relevant template file can be selected, or is selected automatically based on the patient's identity or other inputs (e.g. the patient's condition which was input earlier). In this way, following the completion of the recording, a file comprising the recording data is conveniently and automatically formatted in an email with additional information relevant to the patient's condition without the need to select items for inclusion each time such a patient is seen.

A file comprising recording data capturing the visit may is transmitted to a selected destination. In a primary implementation of the system, the file comprising the recording data is sent as an email attachment to an address of the user's choosing. The email may be automatically formatted to include the user's address. Alternatively, an email build screen may be presented enabling the user to enter their email address and select or enter additional destinations for the message.

In a primary embodiment, the file comprising the recording data is transmitted to a secure electronic storage destination. In this implementation, the email is encrypted and/or securely transmitted to a secure storage database. In one embodiment, the database is a HIPAA-compliant database. In one embodiment, the database is a medical records system. In another primary implementation, a file comprising the recording data is transmitted to the patient, for example, by email, e.g. HIPAA compliant email, or providing a link to a stored copy thereof. This advantageously allows the recipient to replay the visit, and by repeated listening, better understand what was conveyed. Rather than struggling to recall the rich details of the consultation, the patient is afforded an opportunity to replay and better interpret what was conveyed by the provider.

In one embodiment, the patient receives a token, password, or receipt which enables them to access the audio file, for example, order that it be emailed to them, or allowing them to play it back over the telephone, for example, by a dedicated phone line for such access.

In one embodiment, the system is configured to transmit, either in response to a user command or automatically, a file comprising the recording data to a specified destination, for example, a supervising doctor, a customer service department, an insurance company etc, for review of the provider's performance or to create a record.

In one embodiment, by the use of analysis tools, a transcript of the interaction is created by analysis of the recording file data. The transcript file is emailed with the recording file as an email attachment. In one embodiment, the transcript file is emailed or otherwise transmitted to an EMR or EHR service to be included in the subject's medical records. This feature is useful for EMR or EHR systems that do not accommodate storage of audio recordings in a subject's medical record, whereas text data comprising a transcript of the visit can be readily stored in a patient's records in current EMR or EHR systems.

One security feature of the consultation application is a timed-lockout mode wherein, if an input is not made within a certain time interval, the application closes itself and requires a password or biometric input in order to resume operation.

### EXAMPLES

Example 1. Provider-facing VISIT REPLAY™ consultation recording and transmission application. An app named VISIT REPLAY™, for iOS and Android mobile devices was developed. One implementation of the app is a provider-facing version for use by doctors and other practitioners to record office visits and to transmit recording files of the office visit to the patient. This app represents an implementation of the device wherein the consultation recording and transmission application resides entirely on the user's device, and employs a separate communication application comprising an email application to transmit a message wherein a file comprising the recording data is included as an attachment.

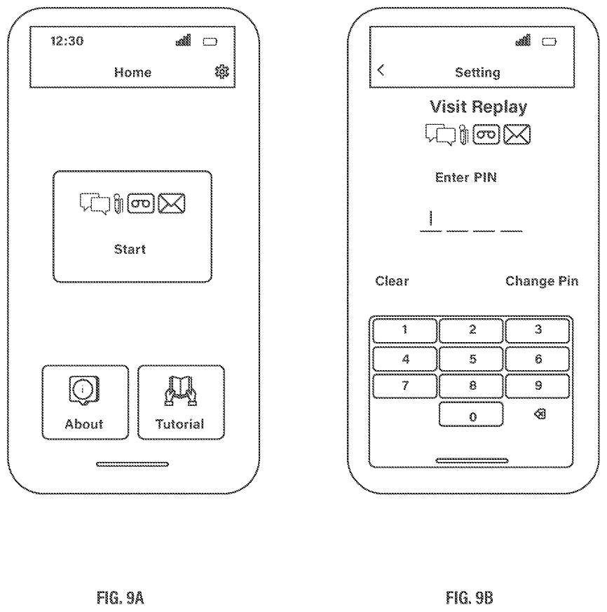

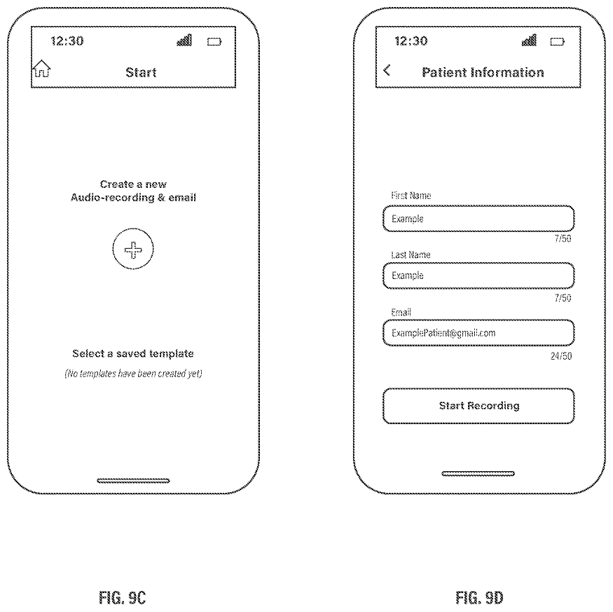

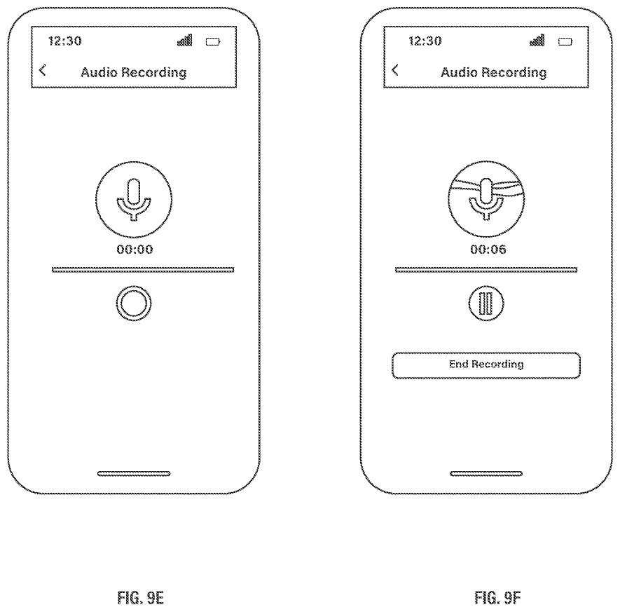

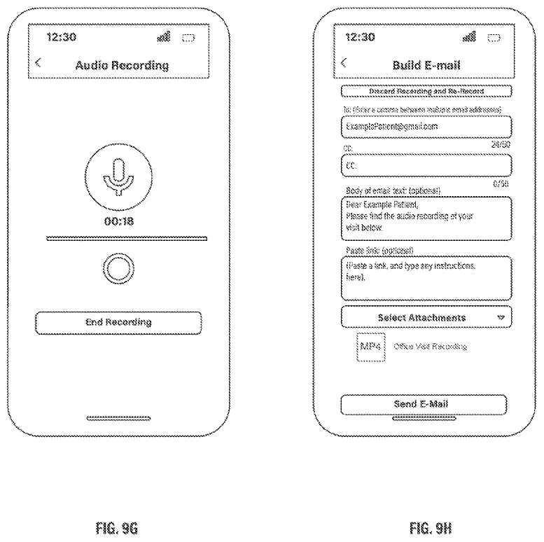

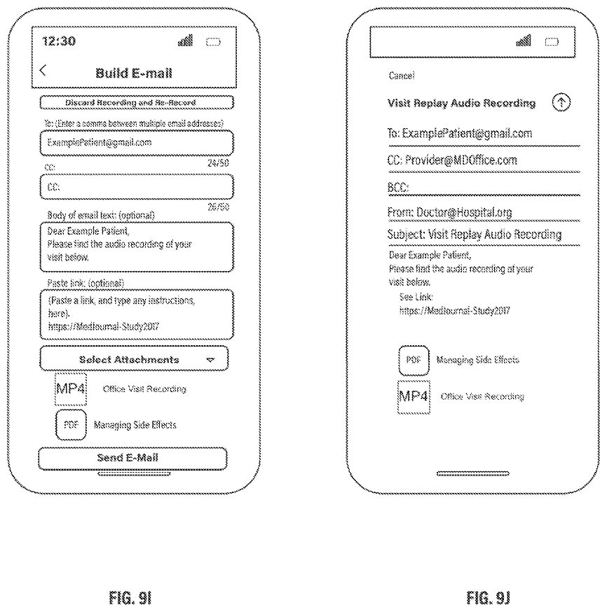

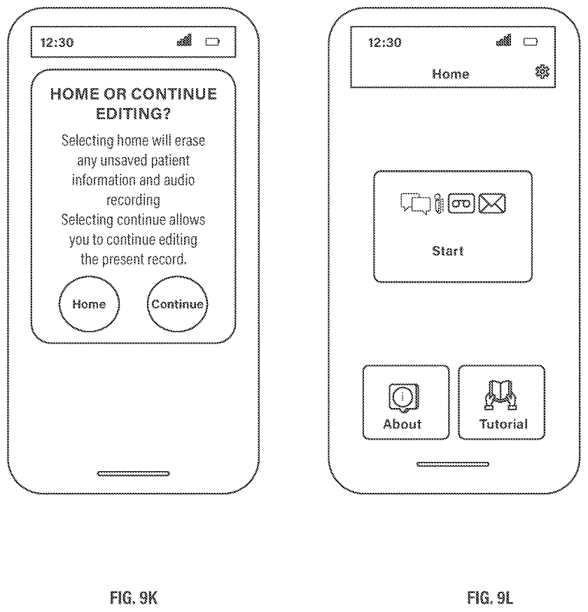

FIG. 9A-9L present exemplary screenshots of the provider-facing VISIT REPLAY™ screens displayed for the user in an embodiment. FIG. 9A depicts the app's home screen, which is displayed for the user when the app is opened. FIG. 9B depicts the password protected entry to the app, wherein the use must enter a password to utilize the application, by the use of a displayed keyboard element comprising a number pad. FIG. 9C depicts a menu page wherein the user may select a button to initiate a new recording and transmission session labeled “Create a new audio recording and email.” The user may also select a button to access a menu of templates for specific types of visits. The ability to make and use email templates allows the user to create specific emails formatted for different types of visits.

Upon the user's selection of the option to create a new audio recording and email, a temporary secure folder is created in the data storage element of the device, and the screen of FIG. 9D is displayed. Here, the user may input data about consultation, in this example, the patient's first and last names, and a recipient email address, for example, the patient's email address. A “start recording” displayed button is presented, and when selected, a new recording file is created in the secure folder and the recording screen of FIG. 9E is displayed. The screen depicted in FIG. 9E presents an audio recording control button for the user. If the user selects the button, the microphone of the device is engaged and commences capturing audio data while the provider consults with the patient, which audio data is written to a recording file. During recording, the screen depicted in FIG. 9F is displayed, presenting a timer, a pause control, and an “end recording” displayed button. Upon the user's selection of the pause recording button, recording is paused, as depicted in FIG. 9G. Upon the user's selection of the end recording displayed button, the “Build email” screen depicted in FIG. 9H is presented. The screen depicted in FIG. 9H presents fillable fields and selectable displayed buttons that allow the user to enter Cc and Bcc email addresses, edit the body of the email, paste links to pertinent materials discussed during the visit, and to select files for attachment to a message. The presented screen includes a pre-populated default message body of the email, which is editable by the user. Depicted in FIG. 91 is the screen after the user has made various inputs. In this example, the user has input a CC email address to themself, a link to pertinent study, and selected a PDF document concerning side effects of an intervention that was discussed. The inputs and selections made here are written to the consultation data file in the secure folder, for example, as text strings for the body of the email and as a file reference or identifier and locator for the attached file. When the user is ready, the user selects a displayed “Send email” button.

Upon the user's selection of the send email displayed button, the consultation application engages the communication application of the device and is directed to create an email message. With the focus of the device now switched to the email communication application, a draft email message is displayed for the user, as depicted in FIG. 9J. The email is populated with data from the consultation data file, including the inputs made by the user in the consultation application, including the subject's email address, the specified CC email address, the web link input by the user, and wherein the PDF file selected by the user for attachment, by the file reference specified in the consultation data file, has been retrieved and included in the message as an attached file. A file comprising the recording data has been attached to the message, in this application it is sent as an M4A file. Upon the user's selection of the displayed “send” icon (up arrow in this example), the email is sent by the email communication application the specified recipients.

The system is configured such that sending of the email changes the focus of the device back to a decision screen in the VISIT REPLAY™ consultation application, as depicted him FIG. 9K. The decision screen presents the user with two options: the option to continue working on the message, or the option to return to the home screen and erase the current session. If the user selects the continue option, they are returned to the “build email” screen depicted in FIG. 9L, and may make changes to the message. If the user selects the “home screen” option, then they are presented with the home screen and the option to create a new recording session, as depicted in FIG. 9M.

Example 2. Patient-facing VISIT REPLAY™ consultation recording and transmission application. A second version of the VISIT REPLAY™ consultation recording and transmission application was developed, in this case configured for the patient. The patient-facing VISIT REPLAY™ works similarly to the provider-facing version of the application. A feature of the patient-facing consultation application is that a notes field is displayed for the user throughout the session, comprising also presented with fillable boxes which enable the user, by a displayed keyboard, to input notes about the consultation, which data is written to the consultation data file. Prompts may suggest the user note who was visited, how they liked the interactions, etc. The fields can also include “follow up” lists of action items, for example, “the doctor told me to get an X-ray” or “the doctor told me to take ibuprofen.” The follow-up entries can automatically bring up links to appropriate service providers, pharmacies, etc. and can also generate automated reminder emails, calendar entries, etc.

The notes field is displayed and accessible to the user throughout the session, including after initiation of a recording session, during the recording capture, and after capture of the recording. Upon the user's command to create a message, an email file is created populated with any notes input by the user in the body of the email message, and a recording data file as an attachment. This enables the user to receive a recording of their consultation with the medical provider and their own notes about the interaction.

Example 3. Service Provider consultation and recording and transmission application. A consultation recording and transmission application for recording an interaction between a service provider and a customer is utilized. A video recording of the interaction is made by engaging the camera and microphone recording elements of the device. Following capture of the interaction, the recording data is transmitted in a message, such an email message, to the service provider and the customer to memorialize the agreed upon scope of work.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A medical consultation analysis system, comprising:
a consultation capture and transmission application comprising:
a client application residing on a computing device; and
a back-end application operating on a remote server;
wherein the computing device includes:
recording hardware elements for capturing consultation audio/audiovisual data;
a user interface configured to display guide materials during consultations; and
network elements for secure data transmission;
a data storage system comprising:
a secure temporary folder on the computing device; and
structured databases on the remote server;
wherein the consultation capture and transmission application is configured to:
initiate recording sessions in response to user commands;
store captured consultation data in encrypted format;
automatically delete consultation data after secure transmission; and
interface with electronic medical record systems;
an analysis application configured to:
transcribe consultation recordings in real-time;
analyze transcribed data using natural language processing;
assess qualitative aspects of communication; and
provide immediate feedback during consultations.

39. The system of claim 38, wherein the analysis application comprises:
automated transcription tools configured to:
convert consultation audio to text in real-time;
identify multiple speakers in conversations;
generate structured transcripts with timestamps; and
export transcripts in multiple formats.

40. The system of claim 38, wherein the analysis application includes:
natural language processing tools configured to:
analyze voice patterns and speech characteristics;
detect emotional indicators in speech;
measure speaking time distributions; and
evaluate communication clarity.

41. The system of claim 38, wherein the analysis application comprises:
performance evaluation tools configured to:
assess provider communication effectiveness;
measure patient engagement levels;
evaluate completeness of information delivery;
identify areas for improvement; and
generate detailed performance reports.

42. The system of claim 38, wherein:
the secure temporary folder is configured to:
store consultation data files in encrypted format;
maintain recording files during transmission; and
automatically delete stored data after confirmation of successful transmission;
the structured databases are configured to:
maintain queryable data formats;
enable secure retrieval of consultation records; and
support distributed processing operations.

43. The system of claim 38, wherein the analysis application provides:
real-time analysis capabilities including:
immediate transcription display;
automated performance metrics;
communication effectiveness indicators; and
interactive feedback during consultations.

44. The system of claim 38, wherein the consultation capture and transmission application:
manages consultation recordings by:
encrypting captured data;
confirming secure transmission;
verifying successful storage; and
executing secure deletion protocols.

45. The system of claim 38, wherein the user interface:
displays guide materials including:
consultation-specific checklists;
communication prompts;
patient comprehension checks; and
real-time analysis feedback.

46. A method of analyzing medical consultations, comprising:
capturing consultation audio/audiovisual data using recording hardware elements;
storing the captured data in a secure temporary folder in encrypted format;
transcribing the consultation data in real-time using automated transcription tools;
analyzing the transcribed data using natural language processing to:
detect speech patterns and characteristics;
measure speaking time distributions; and
evaluate communication effectiveness;
displaying real-time feedback through a user interface during the consultation; and
securely transmitting the analyzed data to electronic medical records.

47. The method of claim 46, further comprising:
displaying consultation-specific guide materials through the user interface;
capturing user inputs regarding consultation progress;
storing the user inputs in encrypted format; and
integrating the user inputs with the analyzed consultation data.

48. The method of claim 46, wherein analyzing the transcribed data comprises:
processing multiple audio streams simultaneously;
identifying distinct speakers in the consultation;
generating structured transcript data with timestamps; and
applying performance evaluation metrics.

49. The method of claim 46, further comprising:
encrypting the captured consultation data;
verifying successful transmission to secure storage;
confirming integration with medical records; and
executing secure deletion protocols.

50. The method of claim 46, wherein displaying real-time feedback comprises:
presenting communication effectiveness indicators;
displaying automated performance metrics;
showing consultation progress tracking; and
providing interactive guidance elements.

51. A method of securing medical consultation data, comprising:
creating a secure temporary folder on a computing device;
capturing consultation recordings using device hardware;
encrypting the captured data using security protocols;
storing the encrypted data in the secure temporary folder;
verifying successful transmission to remote storage; and
automatically executing secure deletion protocols.

52. The method of claim 51, further comprising:
maintaining encrypted data formats during processing;
implementing access controls for temporary storage;
verifying data integrity during transmission; and
confirming complete removal of temporary data.
